(19) United States
(12) Patent Application Publication
(10) Pub. No.: US 2024/0090479 A1
(43) Pub. Date: Mar. 21, 2024

(54) HYPERPROLACTINEMIA OR LACTATION WITHOUT PREGNANCY

(71) Applicant: ABS Global, Inc., DeForest, WI (US)

(72) Inventors: Olivier HIERS, Cottage Grove, WI (US); Jeff BETTHAUSER, Windsor, WI (US); Brian Timothy BURGER, Madison, WI (US); Jessica HAMMERAND, Sun Prairie, WI (US)

(73) Assignee: ABS Global, Inc., DeForest, WI (US)

(21) Appl. No.: 18/262,541

(22) PCT Filed: Jan. 27, 2022
(86) PCT No.: PCT/US22/14042

§ 371 Date: Jul. 21, 2023

Related U.S. Application Data
(60) Provisional application No. 63/199,819, filed on Jan. 27, 2021.

(51) Int. Cl. A01K 67/027; C12N 9/22

(52) U.S. Cl. CPC A01K 67/0275; C12N 9/22; A01K 2227/101; C12N 2310/10

(57) ABSTRACT

Provided are genetically edited animals, particularly dairy animals such as cows or heifers, that lactate without having first been pregnant. Also provided are methods and reagents for creating genetically modified animals. Specifically provided are genetically edited dairy animals that have a histidine to arginine substitution in the prolactin receptor gene.

### CROSS-REFERENCE TO RELATED APPLICATIONS

This application claims the benefit of and priority to U.S. Provisional Patent Application No. US 63/199,819 filed Jan. 27, 2021, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Jan. 26, 2022, is named TD-13-2021-WO1-SEQLST_ST25.txt and is 30,443 bytes in size.

### TECHNICAL FIELD

The present invention relates to a genetically edited non-human mammal. The methods of the present invention provide for modified prolactin receptor (PRLR) genes in mammals so that females produce milk without the need of becoming pregnant.

### BACKGROUND

Hyperprolactinemia is a condition of elevated prolactin levels in blood. The hormone prolactin is secreted from the anterior pituitary gland and is required for induction and maintenance of lactation in the peripartum and postpartum periods. Galactorrhea is commonly caused by hyperprolactinemia. In humans, hyperprolactinemia unrelated to pregnancy occurs in 0.1-0.3% of population. Many of these are caused by drugs or anterior pituitary tumor. However, 10-60% of patients with hyperprolactinemia have normal findings when an MRI is screened for pituitary lesions suggesting a genetic cause.

In cattle, hormone injections have been used to induce the lactation process but yield has been lower than “normal” lactation. Additional injections of bST or growth hormone have increased milk yield, but again, another injection series is required. A genetic solution may allow for natural means of lactation rather than multiple injections of multiple hormones to induce lactation in a non-pregnant animal.

As can be seen, there is a need in the art for animals that produce milk without the need of becoming pregnant.

### SUMMARY

The present invention provides animals that have modulated prolactin receptor (PRLR) expression. The animals have inactivated or otherwise modulated PRLR gene activity resulting in hyperprolactinemia and females that produce milk without the need of becoming pregnant. These animals can be created using any of a number of protocols such as knock-out technology or gene-editing. Thus an embodiment of the invention is a genetically edited or modified animal comprising a genome with modification or inactivation of a PRLR gene. In some embodiments, the modified PRLR protein includes a mutation of His188. Preferably, the modified PRLR protein includes a His188Arg substitution.

Yet another embodiment of the invention is a process of making an animal comprising introducing to a cell or embryo an agent that specifically binds to a chromosomal target site of the cell and causes a double-stranded DNA break or otherwise inactivates a PRLR gene therein using gene editing methods such as the Clustered Regularly Interspaced Short Palindromic Repeats (CRISPR)/Cas system, Transcription Activator-Like Effector Nucleases (TALENs), Zinc Finger Nucleases (ZFN), or recombinase fusion proteins.

Yet another embodiment of the invention is a method of milk production without the need for semen or pregnancy, the method comprising obtaining milk from the genetically edited animals. Hyperprolactinemia in the cattle or other dairy animals reduces or eliminates the need for semen and pregnancy to initiate milk production. Milk production can also begin earlier in the animal lifespan and the methods allow for milking of sub-fertile animals that would otherwise be culled for reproductive reasons.

Also described herein is the use of a PRLR loci in tandem with a polypeptide capable of effecting cleavage and/or integration of specific nucleic acid sequences within the PRLR loci. Examples of the use of PRLR loci in tandem with a polypeptide capable of effecting cleavage and/or integration of the PRLR loci include a polypeptide selected from the group consisting of zinc finger proteins, meganucleases, TAL domains, TALENs, RNA-guided CRISPR/Cas recombinases, leucine zippers, and others known to those in the art. Particular examples include a chimeric (“fusion”) protein comprising a site-specific DNA binding domain polypeptide and cleavage domain polypeptide (e.g., a nuclease), such as a ZFN protein comprising a zinc-finger polypeptide and a FokI nuclease polypeptide. In certain aspects, described herein are polypeptides comprising a DNA-binding domain that specifically binds to a PRLR gene. In some embodiments such a polypeptide may also comprise a nuclease (cleavage) domain or half-domain (e.g., a ZFN, a recombinase, a transposase, or a homing endonuclease, including a homing endonuclease with a modified DNA-binding domain, TAL domains, TALENs, RNA-guided CRISPR/Cas), and/or a ligase domain, such that the polypeptide may induce a targeted double-stranded break, and/or facilitate recombination of a nucleic acid of interest at the site of the break. In particular embodiments, a DNA-binding domain that targets a PRLR locus may be a DNA-cleaving functional domain. The foregoing polypeptides may be used in some embodiments to introduce an exogenous nucleic acid into the genome of a host organism (e.g., an animal species) at one or more PRLR loci.

Further embodiments will become evident from the detailed description of the invention which follows.

### DETAILED DESCRIPTION

The present invention now will be described more fully with reference to the accompanying examples. The invention may be embodied in many different forms and should not be construed as limited to the embodiments set forth in this application; rather, these embodiments are provided so that this disclosure will satisfy applicable legal requirements.

Many modifications and other embodiments of the invention will come to mind to one skilled in the art to which this invention pertains, having the benefit of the teachings presented in the descriptions and the drawings herein. As a result, it is to be understood that the invention is not to be limited to the specific embodiments disclosed and that modifications and other embodiments are intended to be included within the scope of the appended claims. Although specific terms are used in the specification, they are used in a generic and descriptive sense only and not for purposes of limitation.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e. g., Diagnostic Molecular Microbiology: Principles and Applications, D. H. Persing et al., Ed., American Society for Microbiology, Washington, D.C. (1993). The product of amplification is termed an amplicon.

A “binding protein” is a protein that is able to bind to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

As used herein “blastocyst” means an early developmental stage of embryo comprising of inner cell mass (from which embryo proper arises) and a fluid filled cavity typically surrounded by a single layer of trophoblast cells. “Developmental Biology”, sixth edition, ed. by Scott F. Gilbert, Sinauer Associates, Inc., Publishers, Sunderland, Mass. (2000)

The term “Cas” refers to a “CRISPR associated” protein. Non-limiting examples of Cas proteins include Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+and − cleavage half-domains” and “right and left cleavage half-domains” are used interchangeably to refer to pairs of cleavage half-domains that dimerize.

A “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 20070218528, 2008/0131962 and 2011/0201055, incorporated herein by reference in their entireties.

As used herein “conditional knock-out” or “conditional mutation” means when the knock-out or mutation is achieved when certain conditions are met. These conditions include but are not limited to presence of certain inducing agents, recombinases, antibiotics, and certain temperature or salt levels.

The term “conservatively modified variants” applies to both amino acid and nucleic acid sequences. With respect to particular nucleic acid sequences, “conservatively modified variants” refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid.

One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made.

Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another: [1] Alanine (A), Serine (S), Threonine (T); [2] Aspartic acid (D), Glutamic acid (E); [3] Asparagine (N), Glutamine (Q); [4] Arginine (R), Lysine (K); [5] Isoleucine (I), Leucine (L), Methionine (M), Valine (V); and [6] Phenylalanine (F), Tyrosine (Y), Tryptophan (W). See also, Creighton (1984) Proteins W. H. Freeman and Company.

The term “early stage embryo” means any embryo at embryonic stages between fertilized ovum and blastocyst. Typically, eight cell stage and morula stage embryos are referred to as early stage embryos.

By “encoding” or “encoded”, with respect to a specified nucleic acid, is meant comprising the information for translation into the specified protein. A nucleic acid encoding a protein may comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. When the nucleic acid is prepared or altered synthetically, advantage can be taken of known codon preferences of the intended host where the nucleic acid is to be expressed.

“Embryonic stem cells” or “ES cells” means cultured cells derived from inner cell mass of early stage embryo, which are amenable to genetic modification and which retain their totipotency and can contribute to all organs of resulting chimeric animal if injected into host embryo. “Developmental Biology”, sixth edition, ed. by Scott F. Gilbert, Sinauer Associates, Inc., Publishers, Sunderland, Mass. (2000).

As used herein, “fertilization” means the union of male and female gametes during reproduction resulting into formation of zygote, the earliest developmental stage of an embryo.

“Exogenous” refers to a nucleic acid sequence originating outside an organism that has been introduced into the organism. This can refer to sequences which naturally occur in a sexual compatible species, sequences which are synthetic, or sequences from another species.

As used herein “full-length sequence” in reference to a specified polynucleotide or its encoded protein means having the entire amino acid sequence of a native (nonsynthetic), endogenous, biologically active form of the specified protein. Methods to determine whether a sequence is full-length are well known in the art including such exemplary techniques as northern or western blots, primer extension, S1 protection, and ribonuclease protection. Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the bold codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “gene editing,” “gene edited” “genetically edited” and “gene editing effectors” refer to the use of naturally occurring or artificially engineered nucleases, also referred to as “molecular scissors.” The nucleases create specific double-stranded break (DSBs) at desired locations in the genome, which in some cases harnesses the cell's endogenous mechanisms to repair the induced break by natural processes of homologous recombination (HR) and/or nonhomologous end-joining (NHEJ). Gene editing effectors include Zinc Finger Nucleases (ZFNs), Transcription Activator-Like Effector Nucleases (TALENs), the Clustered Regularly Interspaced Short Palindromic Repeats/CAS (CRISPR/Cas) system, and meganuclease re-engineered as homing endonucleases. The terms also include the use of genetic modification procedures and techniques, including, for example, where the change is relatively small and/or does not introduce DNA from a foreign species.

The terms “genetic manipulation” and “genetically manipulated” include gene editing techniques, as well as and/or in addition to other techniques and processes that alter or modify the nucleotide sequence of a gene or gene, or modify or alter the expression of a gene or genes.

As used herein, the terms “guide RNA,” “sgRNA” and “synthetic guide RNA” may be used interchangeably and refer to a single RNA represented as comprising the following elements:
5′-X1-X2-Y-Z-3′

where X1 and X2 represent the crRNA segment, where X1 is the targeting sequence that binds to a portion of a target nucleic acid sequence (e.g., PRLR), X2 is a stem sequence the hybridizes to a tracrRNA, Z represents a tracrRNA segment comprising a nucleotide sequence that is partially or completely complementary to X2, and Y represents a linker sequence. In some embodiments, the linker sequence comprises two or more nucleotides and links the crRNA and tracrRNA segments. In some embodiments, the linker sequence comprises 2, 3, 4, 5, 6, 7, 8, 9, 10 or more nucleotides. In some embodiments, the linker is the loop of the hairpin structure formed when the stem sequence is hybridized with the tracrRNA.

In some embodiments, a synthetic guide RNA is provided as two separate RNAs where (1) one RNA represents a crRNA segment: 5′-X1-X2-3′ where X1 is the targeting sequence that binds to a portion of a target nucleic acid sequence (e.g., PRLR), X2 is a stem sequence the hybridizes to a tracrRNA, and (2) one RNA represents a tracrRNA segment, Z, that is a separate RNA from the crRNA segment and comprises a nucleotide sequence that is partially or completely complementary to X2 of the crRNA. When provided as two separate RNAs, the synthetic guide RNA is formed when the crRNA segment hybridizes to/is complexed with the tracrRNA segment.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

As used herein “homing DNA technology” or “homing technology” covers any mechanisms that allow a specified molecule to be targeted to a specified DNA sequence including Zinc Finger (ZF) proteins, Transcription Activator-Like Effectors (TALEs) meganucleases, and CRISPR systems (e.g., CRISPR/Cas9 systems).

As used herein, “host cell” is in reference to a cell which contains a vector and supports the replication and/or expression of the vector. Host cells may be prokaryotic cells such as E. coli, or eukaryotic cells such as yeast, insect, amphibian, or mammalian cells.

The term “hybridization complex” includes reference to a duplex nucleic acid structure formed by two single-stranded nucleic acid sequences selectively hybridized with each other.

The term “introduced” in the context of inserting a nucleic acid into a cell is equivalent to “transfection” or “transformation” or “transduction,” and includes reference to the incorporation of a nucleic acid into a eukaryotic or prokaryotic cell where the nucleic acid may be incorporated into the genome of the cell (e. g., chromosome, plasmid, plastid or mitochondrial DNA), converted into an autonomous replicon, or transiently expressed (e.g., transfected mRNA).

The term “isolated” refers to material, such as a nucleic acid or a protein, which is: (1) substantially or essentially free from components that normally accompany or interact with it as found in its naturally occurring environment—the isolated material optionally comprises material not found with the material in its natural environment; or (2) if the material is in its natural environment, the material has been synthetically altered by deliberate human intervention to a composition and/or placed at a location in the cell (e.g., genome or subcellular organelle) not native to that material. The alteration to yield the synthetic material can be performed on the material within, or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, the term “knock-in” means replacement of an endogenous gene with a transgene or with same endogenous gene with some structural modification/s, but retaining the transcriptional control of the endogenous gene.

“Knock-out” means disruption of the structure or regulatory mechanism of a gene. Knock-outs may be generated through homologous recombination of targeting vectors, replacement vectors or hit-and-run vectors or random insertion of a gene trap vector resulting into complete, partial or conditional loss of gene function.

As used herein, “localized within the chromosomal region defined by and including” with respect to particular markers includes reference to a contiguous length of a chromosome delimited by and including the stated markers.

As used herein, “marker” includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes of that pair to be followed. A genotype may be defined by use of one or a plurality of markers.

As used herein, “mutation” includes reference to alterations in the nucleotide sequence of a polynucleotide, such as for example a gene or coding DNA sequence (CDS), compared to the wild-type sequence. The term includes, without limitation, substitutions, insertions, frameshifts, deletions, inversions, translocations, duplications, splice-donor site mutations, point-mutations or the like.

As used herein, “nucleic acid” includes reference to a deoxyribonucleotide or ribonucleotide polymer in either single-or double-stranded form, and unless otherwise limited, encompasses conservatively modified variants and known analogues having the essential nature of natural nucleotides in that they hybridize to single-stranded nucleic acids in a manner similar to naturally occurring nucleotides (e. g., peptide nucleic acids).

By “nucleic acid library” is meant a collection of isolated DNA or RNA molecules which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, CA (Berger); Sambrook et al., Molecular Cloning—A Laboratory Manual, 2nded., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary join two protein coding regions, contiguously and in the same reading frame.

As used herein, “polynucleotide” includes reference to a deoxyribopolynucleotide, ribopolynucleotide, or conservatively modified variants; the term may also refer to analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art.

The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms also may apply to conservatively modified variants and to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, the protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not always entirely linear. For instance, polypeptides may be branched as a result of ubiquitization, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. Examples of promoters under developmental control include promoters that preferentially initiate transcription in certain tissues, such as testes, ovaries, or placenta. Such promoters are referred to as “tissue preferred”. Promoters which initiate transcription only in certain tissue are referred to as “tissue specific”. A “cell type” specific promoter primarily drives expression in certain cell types in one or more organs, for example, germ cells in testes or ovaries. An “inducible” or “repressible” promoter is a promoter which is under environmental control. Examples of environmental conditions that may affect transcription by inducible promoters include stress, and temperature. Tissue specific, tissue preferred, cell type specific and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein.

Zinc finger and TALE binding domains can be “engineered” to bind to a predetermined nucleotide sequence, for example via engineering (altering one or more amino acids) of the recognition helix region of naturally occurring zinc finger or TALE proteins. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

A “selected” zinc finger protein or TALE is a protein not found in nature whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970 WO 01/88197, WO 02/099084 and U.S. Publication No. 20110301073.

As used herein, “vector” includes reference to a nucleic acid used in transfection of a host cell and into which can be inserted a polynucleotide. Vectors are often replicons. Expression vectors permit transcription of a nucleic acid inserted therein.

“Wild type” means those animals and blastocysts, embryos or cells derived therefrom, which have not been genetically edited and are usually inbred and outbred strains developed from naturally occurring strains.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

The following terms are used to describe the sequence relationships between a polynucleotide/polypeptide of the present invention with a reference polynucleotide/polypeptide: (a) “reference sequence”, (b) “comparison window”, (c) “sequence identity”, and (d) “percentage of sequence identity”.

(a) As used herein, “reference sequence” is a defined sequence used as a basis for sequence comparison with a polynucleotide/polypeptide of the present invention. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2: 482(1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); and by computerized implementations of these algorithms, including, but not limited to: CLUSTAL in the PC/Gene program by Intelligenetics, Mountain View, California; GAP, BESTFIT, BLAST, FASTA, and TFASTA, and related programs in the GCG Wisconsin Genetics Software Package, Version 10 (available from Accelrys Inc., 9685 Scranton Road, San Diego, California, USA). The CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs that can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215: 403-410 (1990); and, Altschul et al., Nucleic Acids Res. 25: 3389-3402 (1997). Software for performing BLAST analyses is publicly available, for example through the National Center for Biotechnology Information (ncbi.nlm.nih.gov/). This algorithm has been thoroughly described in a number of publications. See, e.g., Altschul SF et al., Gapped BLAST and PSI-BLAST: a new generation of protein database search programs, 25 NUCLEIC ACIDS RES. 3389 (1997); National Center for Biotechnology Information, THE NCBI HANDBOOK [INTERNET], Chapter 16: The BLAST Sequence Analysis Tool (McEntyre J, Ostell J, eds., 2002), available at www(dot)ncbi(dot)nlm(dot)nih(dot)gov/books/NBK21097/pdf/ch16.pdf. The BLASTP program for amino acid sequences has also been thoroughly described (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5877 (1993)). A number of low-complexity filter programs can be employed to reduce such low-complexity alignments. For example, the SEG (Wooten and Federhen, Comput. Chem., 17: 149-163 (1993)) and XNU (Claverie and States, Comput. Chem., 17: 191-201 (1993)) low-complexity filters can be employed alone or in combination.

Unless otherwise stated, nucleotide and protein identity/similarity values provided herein are calculated using GAP (GCG Version 10) under default values. GAP (Global Alignment Program) can also be used to compare a polynucleotide or polypeptide of the present invention with a reference sequence. GAP uses the algorithm of Needleman and Wunsch (J. Mol. Biol. 48: 443-453, 1970) to find the alignment of two complete sequences that maximizes the number of matches and minimizes the number of gaps. GAP represents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff (1989) Proc. Natl. Acad. Sci. USA 89: 10915).

Multiple alignment of the sequences can be performed using the CLUSTAL method of alignment (Higgins and Sharp (1989) CABIOS. 5: 151-153) with the default parameters (GAPPENALTY=10, GAP LENGTH PENALTY=10). Default parameters for pairwise alignments using the CLUSTAL method include KTUPLE 1, GAP PENALTY=3, WINDOW=5 and DIAGONALS SAVED=5.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions may be calculated according to the algorithm of Meyers and Miller, Computer Applic. Biol. Sci., 4: 11-17 (1988), for example as implemented in the program PC/GENE (Intelligenetics, Mountain View, California, USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

### PRLR Gene Editing

The prolactin receptor (PRLR) is a type I cytokine receptor that binds prolactin (PRL) as a transmembrane receptor. Such cytokine receptors have three parts: (1) an extracellular N-terminal portion comprising two fibronectin type III domains, a membrane-distal domain (D1), and a membrane-proximal domain (D2); (2) a single transmembrane α-helical protion and (3) a C-terminal intracellular portion having binding sites for various protein partners involved in intracellular signaling.

PRLR proteins are known and sequences encoding the same are available through Genbank, Ensembl, or other such sources. Bos taurus PRLR nucleic acid and protein sequences are disclosed at Genbank accession numbers NP_776580.1, NP_001034815.1, NM_174155.3, and NM_001039726.2.

In humans, a single mutation in the PRLR causes hyperprolactinemia. The mutation is a heterozygous substitution of A to G at position 635 in the PRLR gene resulting in a His188Arg substitution within the mature receptor. His188 is located within the second fibronectin type III domain of the extracellular N-terminal portion of PRLR. This fibronectin domain type III domain is present at amino acids 105 to 205 of the mature Bos taurus PRLR protein (SEQ ID NO: 3 or 4) and His188 is conserved.

The present disclosure provides a genetically edited animal or animal cell comprising at least one edited chromosomal sequence altering expression of a PRLR protein or other protein associated with hyperprolactinemia. The edited chromosomal sequence may be (1) inactivated, (2) modified, or (3) comprise an integrated sequence. An inactivated chromosomal sequence is altered such that a PRLR protein function is impaired, reduced or eliminated. As used herein, reduced PRLR protein function or activity refers to a reduction in protein function or activity relative to the function of a wild type PRLR protein. Thus, a genetically edited animal comprising an inactivated chromosomal sequence may be termed a “knock out” or a “conditional knock out.” Similarly, a genetically edited animal comprising an integrated sequence may be termed a “knock in” or a “conditional knock in.” Furthermore, a genetically edited animal comprising a modified chromosomal sequence may comprise a targeted point edit(s) or other modification such that an altered protein product is produced. Briefly, the process comprises introducing into an embryo or cell at least one RNA molecule encoding a targeted nuclease and, optionally, at least one accessory polynucleotide. The method further comprises incubating the embryo or cell to allow expression of the nuclease, wherein a double-stranded break introduced into the targeted chromosomal sequence by the nuclease is repaired by a non-homologous end-joining DNA repair process or a homology-directed DNA repair process. The method of editing chromosomal sequences encoding a protein associated with germline development using targeted nuclease technology is rapid, precise, and highly efficient.

In some embodiments of the present invention, a PRLR locus is used as a target site for the site-specific editing. This can include insertion of an exogenous nucleic acid (e.g., a nucleic acid comprising a nucleotide sequence encoding a polypeptide of interest) or deletions of nucleic acids from the locus. In particular embodiments, insertions and/or deletions result in a modified locus. For example, integration of the exogenous nucleic acid and/or deletion of part of the genomic nucleic acid may modify the locus so as to produce a disrupted (i.e., inactivated) PRLR gene.

### Targeted Integration of a Nucleic Acid at a PRLR Locus

Site-specific integration of an exogenous nucleic acid at a PRLR locus may be accomplished by any technique known to those of skill in the art. In some embodiments, integration of an exogenous nucleic acid at a PRLR locus comprises contacting a cell (e.g., an isolated cell or a cell in a tissue or organism) with a nucleic acid molecule comprising the exogenous nucleic acid. In examples, such a nucleic acid molecule may comprise nucleotide sequences flanking the exogenous nucleic acid that facilitate homologous recombination between the nucleic acid molecule and at least one PRLR locus. In particular examples, the nucleotide sequences flanking the exogenous nucleic acid that facilitate homologous recombination may be complementary to endogenous nucleotides of the PRLR locus. In particular examples, the nucleotide sequences flanking the exogenous nucleic acid that facilitate homologous recombination may be complementary to previously integrated exogenous nucleotides.

Integration of a nucleic acid at a PRLR locus may be facilitated (e.g., catalyzed) in some embodiments by endogenous cellular machinery of a host cell, such as, for example and without limitation, endogenous DNA and endogenous recombinase enzymes. In some embodiments, integration of a nucleic acid at a PRLR locus may be facilitated by one or more factors (e.g., polypeptides) that are provided to a host cell. For example, nuclease(s), recombinase(s), and/or ligase polypeptides may be provided (either independently or as part of a chimeric polypeptide) by contacting the polypeptides with the host cell, or by expressing the polypeptides within the host cell. Accordingly, in some examples, a nucleic acid comprising a nucleotide sequence encoding at least one nuclease, recombinase, and/or ligase polypeptide may be introduced into the host cell, either concurrently or sequentially with a nucleic acid to be integrated site-specifically at a PRLR locus, wherein the at least one nuclease, recombinase, and/or ligase polypeptide is expressed from the nucleotide sequence in the host cell.

### DNA-Binding Polypeptides

In some embodiments, site-specific integration may be accomplished by utilizing factors that are capable of recognizing and binding to particular nucleotide sequences, for example, in the genome of a host organism. For instance, many proteins comprise polypeptide domains that are capable of recognizing and binding to DNA in a site-specific manner. A DNA sequence that is recognized by a DNA-binding polypeptide may be referred to as a “target” sequence. Polypeptide domains that are capable of recognizing and binding to DNA in a site-specific manner generally fold correctly and function independently to bind DNA in a site-specific manner, even when expressed in a polypeptide other than the protein from which the domain was originally isolated. Similarly, target sequences for recognition and binding by DNA-binding polypeptides are generally able to be recognized and bound by such polypeptides, even when present in large DNA structures (e.g., a chromosome), particularly when the site where the target sequence is located is one known to be accessible to soluble cellular proteins (e.g., a gene).

While DNA-binding polypeptides identified from proteins that exist in nature typically bind to a discrete nucleotide sequence or motif (e.g., a consensus recognition sequence), methods exist and are known in the art for modifying many such DNA-binding polypeptides to recognize a different nucleotide sequence or motif. DNA-binding polypeptides include, for example and without limitation: zinc finger DNA-binding domains; leucine zippers; UPA DNA-binding domains; GAL4; TAL; LexA; a Tet repressor; LacR; and a steroid hormone receptor.

In some examples, a DNA-binding polypeptide is a zinc finger. Individual zinc finger motifs can be designed to target and bind specifically to any of a large range of DNA sites. Canonical Cys₂His₂ (as well as non-canonical Cys₃His) zinc finger polypeptides bind DNA by inserting an α-helix into the major groove of the target DNA double helix. Recognition of DNA by a zinc finger is modular; each finger contacts primarily three consecutive base pairs in the target, and a few key residues in the polypeptide mediate recognition. By including multiple zinc finger DNA-binding domains in a targeting endonuclease, the DNA-binding specificity of the targeting endonuclease may be further increased (and hence the specificity of any gene regulatory effects conferred thereby may also be increased). See, e.g., Urnov et al. (2005) Nature 435:646-51. Thus, one or more zinc finger DNA-binding polypeptides may be engineered and utilized such that a targeting endonuclease introduced into a host cell interacts with a DNA sequence that is unique within the genome of the host cell.

Preferably, the zinc finger protein is non-naturally occurring in that it is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Selection of target sites; ZFPs and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

In some examples, a DNA-binding polypeptide is a DNA-binding domain from GAL4. GAL4 is a modular transactivator in Saccharomyces cerevisiae, but it also operates as a transactivator in many other organisms. See, e.g., Sadowski et al. (1988) Nature 335:563-4. In this regulatory system, the expression of genes encoding enzymes of the galactose metabolic pathway in S. cerevisiae is stringently regulated by the available carbon source. Johnston (1987) Microbiol. Rev. 51:458-76. Transcriptional control of these metabolic enzymes is mediated by the interaction between the positive regulatory protein, GAL4, and a 17 bp symmetrical DNA sequence to which GAL4 specifically binds (the UAS).

Native GAL4 consists of 881 amino acid residues, with a molecular weight of 99 kDa. GAL4 comprises functionally autonomous domains, the combined activities of which account for activity of GAL4 in vivo. Ma and Ptashne (1987) Cell 48:847-53); Brent and Ptashne (1985) Cell 43(3 Pt 2):729-36. The N-terminal 65 amino acids of GAL4 comprise the GAL4 DNA-binding domain. Keegan et al. (1986) Science 231:699-704; Johnston (1987) Nature 328:353-5. Sequence-specific binding requires the presence of a divalent cation coordinated by 6 Cys residues present in the DNA binding domain. The coordinated cation-containing domain interacts with and recognizes a conserved CCG triplet at each end of the 17 bp UAS via direct contacts with the major groove of the DNA helix. Marmorstein et al. (1992) Nature 356:408-14. The DNA-binding function of the protein positions C-terminal transcriptional activating domains in the vicinity of the promoter, such that the activating domains can direct transcription.

Additional DNA-binding polypeptides that may be utilized in certain embodiments include, for example and without limitation, a binding sequence from a AVRBS3-inducible gene; a consensus binding sequence from a AVRBS3-inducible gene or synthetic binding sequence engineered therefrom (e.g., UPA DNA-binding domain); TAL; LexA (see, e.g., Brent & Ptashne (1985), supra); LacR (see, e.g., Labow et al. (1990) Mol. Cell. Biol. 10:3343-56; Baim et al. (1991) Proc. Natl. Acad. Sci. USA 88(12):5072-6); a steroid hormone receptor (Ellliston et al. (1990) J. Biol. Chem. 265:11517-121); the Tet repressor (U.S. Pat. No. 6,271,341) and a mutated Tet repressor that binds to a tet operator sequence in the presence, but not the absence, of tetracycline (Tc); the DNA-binding domain of NF-κB; and components of the regulatory system described in Wang et al. (1994) Proc. Natl. Acad. Sci. USA 91(17):8180-4, which utilizes a fusion of GAL4, a hormone receptor, and VP16.

In certain embodiments, the DNA-binding domain of one or more of the nucleases used in the methods and compositions described herein comprises a naturally occurring or engineered (non-naturally occurring) TAL effector DNA binding domain. See, e.g., U.S. Patent Publication No. 20110301073, incorporated by reference in its entirety herein.

In other embodiments, the nuclease comprises a CRISPR system (e.g., CRISPR/Cas9 system). The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of Cas genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNAs, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the foreign nucleic acid. Thus, in the bacterial cell, several Cas proteins are involved with the natural function of the CRISPR/Cas system and serve roles in functions such as insertion of the foreign DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Cas9 protein comprises a RuvC nuclease domain and an HNH (H-N-H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and Ill, where domain I is located near the N-terminus of Cas9 and subdomains II and Ill are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA that combines a crRNA and a tracrRNA into a single molecule. The amino acid sequence of a Cas9 protein described herein, as well as certain other Cas proteins herein, may be derived from a Streptococcus (e.g., S. pyogenes, S. pneumoniae, S. thermophilus, S. agalactiae, S. parasanguinis, S. oralis, S. salivarius, S. macacae, S. dysgalactiae, S. anginosus, S. constellatus, S. pseudoporcinus, S. mutans), Listeria (e.g., L. innocua), Spiroplasma (e.g., S. apis, S. syrphidicola), Peptostreptococcaceae, Atopobium, Porphyromonas (e.g., P. catoniae), Prevotella (e.g., P. intermedia), Veillonella, Treponema (e.g., T. socranskii, T. denticola), Capnocytophaga, Finegoldia (e.g., F. magna), Coriobacteriaceae (e.g., C. bacterium), 0/senella (e.g., 0. profusa), Haemophilus (e.g., H. sputorum, H. pittmaniae), Pasteurella (e.g., P. bettyae), 0/ivibacter (e.g., 0. sitiensis), Epilithonimonas (e.g., E. tenax), Mesonia (e.g., M. mobilis), Lactobacillus (e.g., L. plantarum), Bacillus (e.g., B. cereus), Aquimarina (e.g., A. muelleri), Chryseobacterium (e.g., C. palustre), Bacteroides (e.g., B. graminisolvens), Neisseria (e.g., N. meningitidis), Francisella (e.g., F. novicida), or Flavobacterium (e.g., F. frigidarium, F. soli) species, for example. As another example, a Cas9 protein can be any of the Cas9 proteins disclosed in Chylinski et al. (RNA Biology 10:726-737 and U.S. patent application 62/162377, filed May 15, 2015), which are incorporated herein by reference.

Accordingly, the sequence of a Cas9 protein herein can comprise, for example, any of the Cas9 amino acid sequences disclosed in GenBank Accession Nos. G3ECR1 (S. thermophilus), WP_026709422, WP_027202655, WP_027318179, WP_027347504, WP_027376815, WP_027414302, WP_027821588, WP_027886314, WP_027963583, WP_028123848, WP_028298935, Q03J16 (S. thermophilus), EGP66723, EGS38969, EGV05092, EH165578 (S. pseudoporcinus), EIC75614 (S. oralis), EID22027 (S. constellatus), E1169711, EJP22331 (S. oralis), EJP26004 (S. anginosus), EJP30321, EPZ44001 (S. pyogenes), EPZ46028 (S. pyogenes), EQL78043 (S. pyogenes), EQL78548 (S. pyogenes), ERL10511, ERL12345, ERL19088 (S. pyogenes), ESA57807 (S. pyogenes), ESA59254 (S. pyogenes), ESU85303 (S. pyogenes), ETS96804, UC75522, EGR87316 (S. dysgalactiae), EGS33732, EGV01468 (S. oralis), EHJ52063 (S. macacae), EID26207 (S. oralis), EID33364, EIG27013 (S. parasanguinis), EJF37476, EJ019166 (Streptococcus sp. BS35b), EJU16049, EJU32481, YP 006298249, ERF61304, ERK04546, ETJ95568 (S. agalactiae), TS89875, ETS90967 (Streptococcus sp. SR4), ETS92439, EUB27844, (Streptococcus sp. BS21), AFJ08616, EUC82735 (Streptococcus sp. CM6), EWC92088, EWC94390, EJP25691, YP 008027038, YP 008868573, AGM26527, AHK22391, AHB36273, Q927P4, G3ECR1, or Q99ZW2 (S. pyogenes), which are incorporated by reference. A variant of any of these Cas9 protein sequences may be used, but should have specific binding activity, and optionally endonucleolytic activity, toward DNA when associated with an RNA component herein. Such a variant may comprise an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of the reference Cas9. Alternatively, a Cas9 protein may comprise an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to any of the foregoing amino acid sequences, for example. Such a variant Cas9 protein should have specific binding activity, and optionally cleavage or nicking activity, toward DNA when associated with an RNA component herein. A Cas protein herein such as a Cas9 can comprise a heterologous nuclear localization sequence (NLS). A heterologous NLS amino acid sequence herein may be of sufficient strength to drive accumulation of a Cas protein in a detectable amount in the nucleus of a yeast cell herein, for example. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. Non-limiting examples of suitable NLS sequences herein include those disclosed in U.S. Pat. No. 7,309,576, which is incorporated herein by reference.

The Cas endonuclease can comprise a modified form of the Cas9 polypeptide. The modified form of the Cas9 polypeptide can include an amino acid change (e.g., deletion, insertion, or substitution) that reduces the naturally-occurring nuclease activity of the Cas9 protein. For example, in some instances, the modified form of the Cas9 protein has less than 50%, less than 40%, less than 30%, less than 20%, less than 10%, less than 5%, or less than 1% of the nuclease activity of the corresponding wild-type Cas9 polypeptide (US patent application US20140068797 A1, published on Mar. 6, 2014). In some cases, the modified form of the Cas9 polypeptide has no substantial nuclease activity and is referred to as catalytically “inactivated Cas9” or “deactivated cas9 (dCas9).” Catalytically inactivated Cas9 variants include Cas9 variants that contain mutations in the HNH and RuvC nuclease domains. These catalytically inactivated Cas9 variants are capable of interacting with sgRNA and binding to the target site in vivo but cannot cleave either strand of the target DNA.

A catalytically inactive Cas9 can be fused to a heterologous sequence (US patent application US20140068797 A1, published on Mar. 6, 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas9 can also be fused to a FokI nuclease to generate double strand breaks (Guilinger et al. 2014. Nature Biotechnology, 32(6).

In particular embodiments, a DNA-binding polypeptide specifically recognizes and binds to a target nucleotide sequence comprised within a genomic nucleic acid of a host organism. Any number of discrete instances of the target nucleotide sequence may be found in the host genome in some examples. The target nucleotide sequence may be rare within the genome of the organism (e.g., fewer than about 10, about 9, about 8, about 7, about 6, about 5, about 4, about 3, about 2, or about 1 copy(ies) of the target sequence may exist in the genome). For example, the target nucleotide sequence may be located at a unique site within the genome of the organism. Target nucleotide sequences may be, for example and without limitation, randomly dispersed throughout the genome with respect to one another; located in different linkage groups in the genome; located in the same linkage group; located on different chromosomes; located on the same chromosome; located in the genome at sites that are expressed under similar conditions in the organism (e.g., under the control of the same, or substantially functionally identical, regulatory factors); and located closely to one another in the genome (e.g., target sequences may be comprised within nucleic acids integrated as concatemers at genomic loci).

### Targeting Endonucleases

In particular embodiments, a DNA-binding polypeptide that specifically recognizes and binds to a target nucleotide sequence may be comprised within a chimeric polypeptide, so as to confer specific binding to the target sequence upon the chimeric polypeptide. In examples, such a chimeric polypeptide may comprise, for example and without limitation, nuclease, recombinase, and/or ligase polypeptides, as these polypeptides are described above. Chimeric polypeptides comprising a DNA-binding polypeptide and a nuclease, recombinase, and/or ligase polypeptide may also comprise other functional polypeptide motifs and/or domains, such as for example and without limitation: a spacer sequence positioned between the functional polypeptides in the chimeric protein; a leader peptide; a peptide that targets the fusion protein to an organelle (e.g., the nucleus); polypeptides that are cleaved by a cellular enzyme; peptide tags (e.g., Myc, His, etc.); and other amino acid sequences that do not interfere with the function of the chimeric polypeptide.

Functional polypeptides (e.g., DNA-binding polypeptides and nuclease polypeptides) in a chimeric polypeptide may be operatively linked. In some embodiments, functional polypeptides of a chimeric polypeptide may be operatively linked by their expression from a single polynucleotide encoding at least the functional polypeptides ligated to each other in-frame, so as to create a chimeric gene encoding a chimeric protein. In alternative embodiments, the functional polypeptides of a chimeric polypeptide may be operatively linked by other means, such as by cross-linkage of independently expressed polypeptides.

In some embodiments, a DNA-binding polypeptide, or guide RNA that specifically recognizes and binds to a target nucleotide sequence may be comprised within a natural isolated protein (or mutant thereof), wherein the natural isolated protein or mutant thereof also comprises a nuclease polypeptide (and may also comprise a recombinase and/or ligase polypeptide). Examples of such isolated proteins include TALENs, recombinases (e.g., Cre, Hin, Tre, and FLP recombinase), CRISPR systems (e.g., CRISPR/Cas9 systems), and meganucleases.

As used herein, the term “targeting endonuclease” refers to natural or engineered isolated proteins and mutants thereof that comprise a DNA-binding polypeptide or guide RNA and a nuclease polypeptide, as well as to chimeric polypeptides comprising a DNA-binding polypeptide or guide RNA and a nuclease. Any targeting endonuclease comprising a DNA-binding polypeptide or guide RNA that specifically recognizes and binds to a target nucleotide sequence comprised within a PRLR locus (e.g., either because the target sequence is comprised within the native sequence at the locus, or because the target sequence has been introduced into the locus, for example, by recombination) may be utilized in certain embodiments.

Some examples of chimeric polypeptides that may be useful in particular embodiments of the invention include, without limitation, combinations of the following polypeptides: zinc finger DNA-binding polypeptides; a FokI nuclease polypeptide; TALE domains; leucine zippers; transcription factor DNA-binding motifs; and DNA recognition and/or cleavage domains isolated from, for example and without limitation, a TALEN, a recombinase (e.g., Cre, Hin, RecA, Tre, and FLP recombinases), a CRISPR system (e.g., CRISPR/Cas9 system), a meganuclease; and others known to those in the art. Particular examples include a chimeric protein comprising a site-specific DNA binding polypeptide and a nuclease polypeptide. Chimeric polypeptides may be engineered by methods known to those of skill in the art to alter the recognition sequence of a DNA-binding polypeptide comprised within the chimeric polypeptide, so as to target the chimeric polypeptide to a particular nucleotide sequence of interest.

In certain embodiments, the chimeric polypeptide comprises a DNA-binding domain (e.g., zinc finger, TAL-effector domain, etc.) and a nuclease (cleavage) domain. The cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However, any integral number of nucleotides, or nucleotide pairs, can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding, for example, such that one or more exogenous sequences (donors/trangsenes) are integrated at or near the binding (target) sites. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in U.S. Patent Publication No. 20070134796, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474; 20060188987 and 20080131962, the disclosures of all of which are incorporated by reference in their entireties herein.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

### Zinc Finger Nucleases

In specific embodiments, a chimeric polypeptide is a custom-designed zinc finger nuclease (ZFN) that may be designed to deliver a targeted site-specific double-strand DNA break into which an exogenous nucleic acid, or donor DNA, may be integrated (See US Patent publication 20100257638, incorporated by reference herein). ZFNs are chimeric polypeptides containing a non-specific cleavage domain from a restriction endonuclease (for example, FokI) and a zinc finger DNA-binding domain polypeptide. See, e.g., Huang et al. (1996) J. Protein Chem. 15:481-9; Kim et al. (1997a) Proc. Natl. Acad. Sci. USA 94:3616-20; Kim et al. (1996) Proc. Natl. Acad. Sci. USA 93:1156-60; Kim et al. (1994) Proc Natl. Acad. Sci. USA 91:883-7; Kim et al. (1997b) Proc. Natl. Acad. Sci. USA 94:12875-9; Kim et al. (1997c) Gene 203:43-9; Kim et al. (1998) Biol. Chem. 379:489-95; Nahon and Raveh (1998) Nucleic Acids Res. 26:1233-9; Smith et al. (1999) Nucleic Acids Res. 27:674-81. In some embodiments, the ZFNs comprise non-canonical zinc finger DNA binding domains (see US Patent publication 20080182332, incorporated by reference herein). The FokI restriction endonuclease must dimerize via the nuclease domain in order to cleave DNA and introduce a double-strand break. Consequently, ZFNs containing a nuclease domain from such an endonuclease also require dimerization of the nuclease domain in order to cleave target DNA. Mani et al. (2005) Biochem. Biophys. Res. Commun. 334:1191-7; Smith et al. (2000) Nucleic Acids Res. 28:3361-9. Dimerization of the ZFN can be facilitated by two adjacent, oppositely oriented DNA-binding sites. Id.

In particular examples, a method for the site-specific integration of an exogenous nucleic acid into at least one PRLR locus of a host comprises introducing into a cell of the host a ZFN, wherein the ZFN recognizes and binds to a target nucleotide sequence, wherein the target nucleotide sequence is comprised within at least one PRLR locus of the host. In certain examples, the target nucleotide sequence is not comprised within the genome of the host at any other position than the at least one PRLR locus. For example, a DNA-binding polypeptide of the ZFN may be engineered to recognize and bind to a target nucleotide sequence identified within the at least one PRLR locus (e.g., by sequencing the PRLR locus). A method for the site-specific integration of an exogenous nucleic acid into at least one PRLR locus of a host that comprises introducing into a cell of the host a ZFN may also comprise introducing into the cell an exogenous nucleic acid, wherein recombination of the exogenous nucleic acid into a nucleic acid of the host comprising the at least one PRLR locus is facilitated by site-specific recognition and binding of the ZFN to the target sequence (and subsequent cleavage of the nucleic acid comprising the PRLR locus).

### Optional Exogenous Nucleic Acids for Integration at a PRLR Locus

Embodiments of the invention may include one or more nucleic acids selected from the group consisting of: an exogenous nucleic acid for site-specific integration in at least one PRLR locus, for example and without limitation, an ORF; a nucleic acid comprising a nucleotide sequence encoding a targeting endonuclease; and a vector comprising at least one of either or both of the foregoing. Thus, particular nucleic acids for use in some embodiments include nucleotide sequences encoding a polypeptide, structural nucleotide sequences, and/or DNA-binding polypeptide recognition and binding sites.

### Optional Exogenous Nucleic Acid Molecules for Site-Specific Integration

As noted above, insertion of an exogenous sequence (also called a “donor sequence” or “donor”) is provided, for example for expression of a polypeptide, correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient homology directed repair (HDR) at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded or double-stranded and can be introduced into a cell in linear or circular form. See e.g., U.S. Patent Publication Nos. 20100047805, 20110281361, 20110207221 and U.S. application Ser. No. 13/889,162. If introduced in linear form, the ends of the donor sequence can be protected (e.g. from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally integrated so that its expression is driven by the endogenous promoter at the integration site, namely the promoter that drives expression of the endogenous gene into which the donor is integrated (e.g., PRLR). However, it will be apparent that the donor may comprise a promoter and/or enhancer, for example a constitutive promoter or an inducible or tissue specific promoter.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

Exogenous nucleic acids that may be integrated in a site-specific manner into at least one PRLR locus, so as to modify the PRLR locus, in embodiments include, for example and without limitation, nucleic acids comprising a nucleotide sequence encoding a polypeptide of interest; nucleic acids comprising an agronomic gene; nucleic acids comprising a nucleotide sequence encoding an RNAi molecule; or nucleic acids that disrupt the PRLR gene.

In some embodiments, an exogenous nucleic acid is integrated at a PRLR locus, so as to modify the PRLR locus, wherein the nucleic acid comprises a nucleotide sequence encoding a polypeptide of interest, such that the nucleotide sequence is expressed in the host from the PRLR locus. In some examples, the polypeptide of interest (e.g., a foreign protein) is expressed from a nucleotide sequence encoding the polypeptide of interest in commercial quantities. In such examples, the polypeptide of interest may be extracted from the host cell, tissue, or biomass.

### Nucleic Acid Molecules Comprising a Nucleotide Sequence Encoding a Targeting Endonuclease

In some embodiments, a nucleotide sequence encoding a targeting endonuclease may be engineered by manipulation (e.g., ligation) of native nucleotide sequences encoding polypeptides comprised within the targeting endonuclease. For example, the nucleotide sequence of a gene encoding a protein comprising a DNA-binding polypeptide may be inspected to identify the nucleotide sequence of the gene that corresponds to the DNA-binding polypeptide, and that nucleotide sequence may be used as an element of a nucleotide sequence encoding a targeting endonuclease comprising the DNA-binding polypeptide. Alternatively, the amino acid sequence of a targeting endonuclease may be used to deduce a nucleotide sequence encoding the targeting endonuclease, for example, according to the degeneracy of the genetic code.

In exemplary nucleic acid molecules comprising a nucleotide sequence encoding a targeting endonuclease, the last codon of a first polynucleotide sequence encoding a nuclease polypeptide, and the first codon of a second polynucleotide sequence encoding a

DNA-binding polypeptide, may be separated by any number of nucleotide triplets, e.g., without coding for an intron or a “STOP.” Likewise, the last codon of a nucleotide sequence encoding a first polynucleotide sequence encoding a DNA-binding polypeptide, and the first codon of a second polynucleotide sequence encoding a nuclease polypeptide, may be separated by any number of nucleotide triplets. In these and further embodiments, the last codon of the last (i.e., most 3′ in the nucleic acid sequence) of a first polynucleotide sequence encoding a nuclease polypeptide, and a second polynucleotide sequence encoding a DNA-binding polypeptide, may be fused in phase-register with the first codon of a further polynucleotide coding sequence directly contiguous thereto, or separated therefrom by no more than a short peptide sequence, such as that encoded by a synthetic nucleotide linker (e.g., a nucleotide linker that may have been used to achieve the fusion). Examples of such further polynucleotide sequences include, for example and without limitation, tags, targeting peptides, and enzymatic cleavage sites. Likewise, the first codon of the most 5′ (in the nucleic acid sequence) of the first and second polynucleotide sequences may be fused in phase-register with the last codon of a further polynucleotide coding sequence directly contiguous thereto, or separated therefrom by no more than a short peptide sequence.

A sequence separating polynucleotide sequences encoding functional polypeptides in a targeting endonuclease (e.g., a DNA-binding polypeptide and a nuclease polypeptide) may, for example, consist of any sequence, such that the amino acid sequence encoded is not likely to significantly alter the translation of the targeting endonuclease. Due to the autonomous nature of known nuclease polypeptides and known DNA-binding polypeptides, intervening sequences will not in examples interfere with the respective functions of these structures.

### Other Knockout Methods

Various other techniques known in the art can be used to inactivate genes to make knock-out animals and/or to introduce nucleic acid constructs into animals to produce founder animals, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA 82, 6148-1652), gene targeting into embryonic stem cells (Thompson et al. (1989) Cell 56, 313-321), electroporation of embryos (Lo (1983) Mol. Cell. Biol. 3, 1803-1814), sperm-mediated gene transfer (Lavitrano et al. (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374). Pronuclear microinjection, sperm mediated gene transfer, and somatic cell nuclear transfer are particularly useful techniques. An animal that is genomically edited is an animal wherein all of its cells have the genetic edit, including its germ line cells. When methods are used that produce an animal that is mosaic in its genetic edit, the animals may be bred and progeny that are genomically edited may be selected. Cloning, for instance, may be used to make a mosaic animal if its cells are modified at the blastocyst state, or genomic editing can take place when a single-cell is edited. Animals that are edited so that they produce milk without being pregnant can be homozygous or heterozygous for the modification, depending on the specific approach that is used. If a particular gene is inactivated by a knock out modification, homozygosity would normally be required. If a particular gene is inactivated by an RNA interference or dominant negative strategy, then heterozygosity is often adequate.

Typically, in embryo/zygote microinjection, a nucleic acid construct is introduced into a fertilized egg; 1-cell fertilized eggs are used as the pronuclei containing the genetic material from the sperm head and the egg can be made visible within the cytoplasm or ooplasm. Pronuclear staged fertilized eggs can be obtained in vitro or in vivo (i.e., surgically recovered from the oviduct of donor animals). In vitro fertilized eggs can be produced as follows. For example, bovine ovaries can be collected at an abattoir, and maintained at 30° C. during transport. Ovaries can be washed and isolated for follicular aspiration, and follicles ranging from 3-10 mm can be aspirated into 50 mL conical centrifuge tubes using 18 gauge needles and under vacuum. Follicular fluid and aspirated oocytes can be rinsed through pre-filters with commercial TL-HEPES (ABT 360, Pullman, WA). Oocytes surrounded by a compact cumulus mass can be selected and placed into OOCYTE MATURATION MEDIUM TCM-199 (Gibco) supplemented with Luteinizing hormone (10 IU/ml; Sigma), estradiol (1 mg/ml; Sigma), and FBS (10%; GE Hyclone, Logan, UT) at 38.58 C in a humidified 5% CO2 incubator.

Mature oocytes (10) can be fertilized in 50 ill BO-IVF media (IVF Bioscience, Falmouth UK). In preparation for in vitro fertilization (IVF), previously frozen bull sperm can be thawed and remove the semen extender by placing semen on a column of 80% BoviPure (Spectrum Technologies) and centrifuge for 15 minutes at 500 g. The sperm pellet can be resuspended in TL Hepes and centrifuged again for 5 minutes at 300 g. The washed sperm pellet can be resuspended in IVF media at a concentration of 10×10⁶ cells/mL and used to fertilize the mature oocytes by adding 1×10⁴ sperm/oocyte. Co-incubation of sperm and oocytes can be performed in an incubator at 38.5 C and 6% CO2. After 16 hours of co-incubation, fertilized oocytes (zygotes) can be stripped free of the cumulus cells by vortexing for up to 3 minutes.

Linearized nucleic acid constructs, sgRNP or mRNA can be injected into one of the pronuclei or into the cytoplasm. Then the injected eggs can be incubated in BO-IVC media (IVF Bioscience) for seven days to develop into the blastocyst embryo stage. If performing pronucleus injection, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation.

Embryos can be non-surgically transferred into uteri of synchronous or asynchronous recipient heifers or cows. Typically, one embryo will be deposited into the uterine horn of an estrus synchronized recipient. Twenty-eight to 42 days after fertilization, an ultrasound exam can confirm if the recipient is pregnant.

In somatic cell nuclear transfer, a gene-edited cell (e.g., a gene-edited bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the gene edited cell into the perivitelline space of an enucleated oocyte arrested at meiosis 2. In some conventions, oocytes arrested at meiosis-2 are termed eggs. After producing a bovine embryo (e.g., by fusing the gene edited cell and enucleated oocyte and subsequently activating the oocyte), the embryo is cultured in BO-IVC for seven days to reach the blastocyst stage and then non-surgically transferred to the uterus of an estrus synchronized recipient. See, for example, Cibelli et al. (1998) Science 280, 1256-1258 and U.S. Pat. No. 6,548,741.

Standard breeding techniques can be used to create animals that are homozygous for the desired edit from the initial heterozygous founder animals. Homozygosity may not be required, however. Genetically edited animals described herein can be bred with other animals of interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Genetically edited cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, genetically modified animals can be produced that are not constrained to retain selectable markers in subsequent generations.

Once genetically modified animals have been generated, expression of a nucleic acid can be assessed using standard techniques. Initial screening can be accomplished by Illumina sequencing or Southern blot analysis to determine whether or not an edit has taken place.

Expression of a nucleic acid sequence encoding a polypeptide in the tissues of genetically modified animals can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) systems are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of inactivating a gene in a genetically edited animal comprises inducing RNA interference against a target gene and/or nucleic acid such that expression of the target gene and/or nucleic acid is reduced.

For example, the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al. (1998) Nature 391:806; Romano and Masino (1992) Mol. Microbiol. 6:3343; Cogoni et al. (1996) EMBO J. 15:3153; Cogoni and Masino (1999) Nature 399:166; Misquitta and Paterson (1999) Proc. Natl. Acad. Sci. USA 96:1451; and Kennerdell and Carthew (1998) Cell 95:1017. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically edited animal such as a livestock animal that expresses an RNAi directed against a PRLR gene. The RNAi may be, for instance, selected from the group consisting of siRNA, shRNA, dsRNA, and miRNA.

### Inducible Systems

An inducible system may be used to control expression of a PRLR gene. Various inducible systems are known that allow spatiotemporal control of expression of a gene. Several have been proven to be functional in vivo in animal systems.

An example of an inducible system is the tetracycline (tet)-on promoter system, which can be used to regulate transcription of the nucleic acid. In this system, a mutated Tet repressor (TetR) is fused to the activation domain of herpes simplex virus VP 16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically edited animals. One animal line expresses the activator (tTA, rtTA, or Cre recombinase) under the control of a selected promoter. Another set of genetically modified animals express the acceptor, in which the expression of the gene of interest (or the gene to be modified) is under the control of the target sequence for the tTA/rtTA transactivators (or is flanked by loxP sequences). Mating the two animal lines provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed foxed DNA) is excised by Cre-mediated recombination. Control of Cre expression in a genetically modified animal, using either spatial control (with a tissue- or cell-specific promoter), or temporal control (with an inducible system), results in control of DNA excision between the two loxP sites. One application is for conditional gene inactivation (conditional knockout). Another approach is for protein over-expression, wherein a foxed stop codon is inserted between the promoter sequence and the DNA of interest. Genetically edited animals do not express the modified gene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically edited animal such as a dairy animal that comprise a PRLR gene that is under control of an inducible system. The genetic modification of an animal may be genomic or mosaic. The inducible system may be, for instance, selected from the group consisting of Tet-On, Tet-Off, Cre-lox, and Hif1 alpha.

### Vectors and Nucleic Acids

A variety of nucleic acids may be introduced into cells for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller (1997) Antisense Nucleic Acid Drug Dev. 7(3):187; and Hyrup et al. (1996) Bioorgan. Med. Chem. 4:5. In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, inducible promoters, and promoters responsive or unresponsive to a particular stimulus. Suitable tissue specific promoters can result in preferential expression of a nucleic acid transcript in beta cells and include, for example, the human insulin promoter. Other tissue specific promoters can result in preferential expression in, for example, hepatocytes or heart tissue and can include the albumin or alpha-myosin heavy chain promoters, respectively. In other embodiments, a promoter that facilitates the expression of a nucleic acid molecule without significant tissue or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al. (2001) Hum. Gene Ther. 12:563; and Kiwaki et al. (1996) Hum. Gene Ther. 7:821.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. (1992) 89:6861, for a review of Cre/lox technology, and Brand and Dymecki, Dev. Cell (2004) 6:7. A transposon containing a Cre- or Flp-activatable transgene interrupted by a selectable marker gene also can be used to obtain genetically modified animals with conditional expression of a transgene. For example, a promoter driving expression of the marker/transgene can be either ubiquitous or tissue-specific, which would result in the ubiquitous or tissue-specific expression of the marker in F0 animals (e.g., bovine). Tissue specific activation of the transgene can be accomplished, for example, by crossing an animal that ubiquitously expresses a marker-interrupted transgene to an animal expressing Cre or Flp in a tissue-specific manner, or by crossing an animal that expresses a marker-interrupted transgene in a tissue-specific manner to an animal that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be methylated using an SssI CpG methylase (New England Biolabs, Ipswich, Mass.). In general, the nucleic acid construct can be incubated with S-adenosylmethionine and Sssl CpG-methylase in buffer at 37° C. Hypermethylation can be confirmed by incubating the construct with one unit of HinPlI endonuclease for 1 hour at 37° C. and assaying by agarose gel electrophoresis.

Nucleic acid constructs can be introduced into embryonic, fetal, or adult animal cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. (2003) Nucleic Acids Res. 31:6873); Tol2 (Kawakami (2007) Genome Biology 8(Suppl.1):57; Minos (Pavlopoulos et al. (2007) Genome Biology 8(Suppl.1):S2); Hsmarl (Miskey et al. (2007)) Mol Cell Biol. 27:4589); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Insulator elements also can be included in a nucleic acid construct to maintain expression of the exogenous nucleic acid and to inhibit the unwanted transcription of host genes. See, for example, U.S. Publication No. 2004/0203158. Typically, an insulator element flanks each side of the transcriptional unit and is internal to the inverted repeat of the transposon. Non-limiting examples of insulator elements include the matrix attachment region-(MAR) type insulator elements and border-type insulator elements. See, for example, U.S. Pat. Nos. 6,395,549, 5,731,178, 6,100,448, and 5,610,053, and U.S. Publication No. 2004/0203158.

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand).

### Founder Animals, Traits, and Reproduction

Founder animals may be produced by cloning and other methods described herein. The founders can be homozygous for a genetic modification, as in the case where a zygote or a primary cell undergoes a homozygous modification. Similarly, founders can also be made that are heterozygous. In the case of PRLR knockouts, the founders are preferably heterozygous. The founders may be genomically modified, meaning that all of the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified.

In livestock, many alleles are known to be linked to various traits such as production traits, type traits, workability traits, and other functional traits. Practitioners are accustomed to monitoring and quantifying these traits, e.g., Visscher et al., Livestock Production Science, 40 (1994) 123-137, U.S. Pat. No. 7,709,206, US 2001/0016315, US 2011/0023140, and US 2005/0153317. An animal may include a trait chosen from a trait in the group consisting of a production trait, a type trait, a workability trait, a fertility trait, a mothering trait, and a disease resistance trait. Further traits include expression of a recombinant gene product. Animals with a desired trait or traits may be modified to produce milk without the need of becoming pregnant.

### Administration of Hormones or Drugs to Artificially Induce Lactation or Increase Milk Yield

In certain embodiments, a hormone or drug to artificially induce lactation or to further increase milk production may be administered to the genetically edited mammals. In other embodiments, the genetically edited mammals produce milk without being pregnant and without the use of a hormone or drug to artificially induce lactation. Methods to artificially induce lactation include, for example, use of exogenous hormones such as estrogen and progesterone, either alone or in combination, glucocorticoids including dexamethasone, and the drug reserpine.

Estrogen initiates lactation by causing release of prolactin from the anterior pituitary gland into the blood and increasing the number of prolactin receptors in mammary cells. Glucocorticoids displace progesterone from mammary cell receptors, thereby reducing the progesterone block to prolactin receptor synthesis. Exogenous dexamethasone, a synthetic glucocorticoid, can also mimic increased glucocorticoid levels. Reserpine causes prolactin release and increases blood prolactin levels.

Bovine somatotropin (bST), also known as bovine growth hormone (BGH), is used to increase milk production in dairy cows. Somatotropin is a protein hormone produced in the pituitary gland of animals and is essential for normal growth, development, and health maintenance and can be found naturally in milk. The hormone is periodically injected into cows and makes the mammary glands of dairy cows take in more nutrients from the bloodstream. Administration of bST to dairy cows can improve the efficiency of milk synthesis and increase milk production.

### Kits

The present disclosure also provides kits for generating a nonhuman mammal that produces milk without being pregnant. In one aspect, the kits of the present technology comprise a gRNA that specifically hybridizes to a prolactin receptor (PRLR) gene, a homology directed repair (HDR) template, and instructions for use. In some embodiments, the gRNA comprises a sequence of any one of SEQ ID NO: 9-13 or any complement thereof. Additionally or alternatively, in some embodiments of the kits disclosed herein, the HDR template comprises a sequence of any one of SEQ ID NOs: 14-17 or any complement thereof. Optionally, the above described components of the kits of the present technology are packed in suitable containers and labeled for the generation of a nonhuman mammal that produces milk without being pregnant.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the reagents towards a higher volume. Furthermore, the kit may comprise instructions for diluting the reagents and/or instructions for using the reagents, whether diluted or not. The containers may be formed from a variety of materials such as glass or plastic and may have a sterile access port (for example, the container may be an intravenous solution bag or a vial having a stopper which may be pierced by a hypodermic injection needle). The kit may further include other materials desirable from a commercial and user standpoint, including other buffers, diluents, filters, needles, syringes, culture medium for one or more of the suitable hosts. The kits may optionally include instructions customarily included in commercial packages of products that contain information about, for example, the indications, usage, dosage, manufacture, administration, contraindications and/or warnings concerning the use of such products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### EXAMPLES

### Example 1: Design, Construction, and Testing of Bovine PRLR CRISPR Reagents

The inventors identified NGG PAMs 20 bp up- and downstream of the codon for His188 and designed gRNAs based on the corresponding spacer sequences (Table 1).

TABLE 1

| Guide | Position | Strand | Sequence | PAM |
|---|---|---|---|---|
| PRLR_H188R_1 | 123355 | 1 | ATTCGCTGCAAGCCAGACCA (SEQ ID NO: 9) | TGG |
| (used in | | | | |
| SpyCas9_1) | | | | |
| PRLR_H188R_2 | 123356 | −1 | TCACTCCAGTATCCATGGTC (SEQ ID NO: 10) | TGG |
| (used in | | | | |
| SpyCas9_2) | | | | |
| PRLR_H188R_3 | 123361 | −1 | TCCACTCACTCCAGTATCCA (SEQ ID NO: 11) | TGG |
| (used in | | | | |
| SpyCas9_3) | | | | |
| PRLR_H188R_4 | 123362 | 1 | GCAAGCCAGACCATGGATAC (SEQ ID NO: 12) | TGG |
| (used in | | | | |
| SpyCas9_4, or | | | | |
| Stherm_4) | | | | |
| PRLR_H188R_5 | 123371 | 1 | ACCATGGATACTGGAGTGAG (SEQ ID NO: 13) | TGG |
| (used in | | | | |
| SpyCas9_5, or | | | | |
| Stherm_5) | | | | |

The skilled person in the art would recognize that mRNA equivalents of PRLR gRNAs (e.g., SEQ ID NOs: 9-13), i.e., where uracil replaces one or more thymine nucleotides within the gRNA, are contemplated as falling within the scope of the present technology.

The gRNAs were ordered from IDT as Alt-R CRISPR-Cas9 sgRNAs, complexed with Spy Cas9 or Stherm Cas9 protein to form sgRNPs, and nucleofected into bovine embryonic fibroblasts (BEFs). Approximately 48 hrs post-nucleofection, gDNA was extracted from cells and sequenced at the target site by Illumina sequencing. The percentage of Illumina reads containing insertions and deletions (indels) at the SpyCas9 cut site serves as a proxy for cutting efficiency, where sgRNAs that result in a large number of indels at the target site are considered to have high cutting efficiency.

The gRNAs demonstrating high cutting efficiency were tested for their ability to facilitate conversion of His188 to arginine, using single-stranded oligonucleotide donors (ssODNs) as template for HDR-mediated repair. In order to optimize HDR-mediated repair frequency, we tested ssODNs with either 50 nt or 75 nt homology arms, with homology to both target and non-target strands. All ssODNs tested were symmetric around the codon replacement site and contained three phosphorothioate linkages on the 3′ and 5′ ends to prevent intracellular nuclease degradation.

The sequences of the HDR repair templates used in the experiments are provided below:

50 nucleotide arms-top strand (50T):
(SEQ ID NO: 14)
CTTATATCCAGGACAAAAATACCTTGTGCAGATTCGCTGCAAGCCAGAC
CGTGGATACTGGAGTGAGTGGAGCCCAGAGAGCTCCATCCAGATACCTA
ATG
50 nucleotide arms-bottom strand (50B):
(SEQ ID NO: 15)
CATTAGGTATCTGGATGGAGCTCTCTGGGCTCCACTCACTCCAGTATCC
ACGGTCTGGCTTGCAGCGAATCTGCACAAGGTATTTTTGTCCTGGATAT
AAG
75 nucleotide arms-top strand (75T):
(SEQ ID NO: 16)
AGCAAACTCAGCTTAAGATTTTCAACTTATATCCAGGACAAAAATACCT
TGTGCAGATTCGCTGCAAGCCAGACCGTGGATACTGGAGTGAGTGGAGC
CCAGAGAGCTCCATCCAGATACCTAATGGTAAGTGTCTTGAGATTCCCC
AAGG
75 nucleotide arms-top strand (75B):
(SEQ ID NO: 17)
CCTTGGGGAATCTCAAGACACTTACCATTAGGTATCTGGATGGAGCTCT
CTGGGCTCCACTCACTCCAGTATCCACGGTCTGGCTTGCAGCGAATCTG
CACAAGGTATTTTTGTCCTGGATATAAGTTGAAAATCTTAAGCTGAGTT
TGCT

Table 2 shows the average editing efficiency of the PRLR_H188 gRNAs based on the corresponding spacer sequences of SEQ ID Nos: 9-13 in the absence of HDR template.

TABLE 2

| sgRNA-Cas9 | Average % edited |
|---|---|
| SpyCas9_1 | 98 |
| SpyCas9_2 | 97 |
| SpyCas9_3 | 98 |
| SpyCas9_4 | 98 |
| SpyCas9_5 | 98 |
| Stherm_4 | 99 |
| Stherm_5 | 98 |

As shown in Table 2, the average editing efficiency of the PRLR_H188 gRNAs disclosed in Table 1 ranged from 97%-99% in the absence of HDR template.

Table 3 shows the average percentage of correct A-to-G editing of the PRLR_H188 gRNAs based on the corresponding spacer sequences of SEQ ID NOs: 9-13 in the presence or absence of HDR templates.

TABLE 3

| | | HDR | | | | | | | |
|---|---|---|---|---|---|---|---|---|---|
| Concentration | | Template | SpyCas9_1 | SpyCas9_2 | SpyCas9_3 | SpyCas9_4 | SpyCas9_5 | Stherm_4 | Stherm_5 |
| 0 | pmol | None | 0 | 0.1 | 0 | 0 | 0 | 0 | 0 |
| 12.5 | pmol | 50T | 27.7 | 51.2 | 1.0 | 43.9 | 0.2 | 53.8 | 0.5 |
| | | 50B | 33.3 | 42.7 | 0.2 | 27.0 | 0.1 | 31.9 | 0.2 |
| | | 75T | 28.7 | 48.0 | 0.9 | 37.0 | 0.2 | 47.9 | 0.6 |
| | | 75B | 18.8 | 35.6 | 0.1 | 14.1 | 0.6* | 13.9 | 0.0 |
| 25 | pmol | 50T | 31.8 | 49.8 | 0.8 | 41.0 | 0.3 | 55.2** | 0.6 |
| | | 50B | 28.7 | 44.3* | 0.4 | 27.2 | 0.5 | 17.8 | 0.1 |
| | | 75T | 27.7 | 48.9* | 3.4 | 40.4 | 0.3 | 46.5 | 0.7* |
| | | 75B | 27.0 | 34.9 | 0.2 | 20.6 | 0.3 | 27.8 | 0.1 |

*Dropout
**2 Dropouts

Table 3 shows the average percentage of correct A-to-G editing of the PRLR_H188 gRNAs disclosed in Table 1 in the presence of 12.5 pmol and 25 pmol HDR templates. PRLR_H188R_2 (SEQ ID NO: 10) and PRLR_H188R_4 (SEQ ID NO: 12) showed 18%-55% correct A-to-G editing in the presence of 25 pmol of all tested HDR templates. Moreover, Table 3 demonstrates that 50 bp arm sgRNAs exhibited a higher degree of correct A-to-G editing compared with the 75 bp arm counterparts, and that top strand sgRNAs exhibited a higher degree of correct A-to-G editing compared with the bottom strand counterparts.

### Example 2: The CRISPR/Cas System for Genetic Editing

Following successful validation in cell culture, both Cas9 protein and sgRNA targeting PRLR will be injected into 1-cell zygotes using an Eppendorf Femtojet injector (see Example 5 for an exemplary protocol). The injected embryos will be allowed to 15 progress to blastocyst stage, DNA will be collected, and sequenced by Illumina sequencing.

### Example 3: Screening and Breeding of Animals

Liveborn animals following transfer of edited embryos will be genotyped as follows. Tissue samples (for example hair follicle, ear notches, tail clips, blood) will be taken and DNA extracted. The PRLR gene will be amplified using suitable primers and sequenced by Illumina sequencing. Animals will be characterized as non-edited, heterozygous edited or homozygous edited.

Heterozygous and homozygous PRLR edited females will be monitored for hyperprolactinemia and lactation without pregnancy. A supply of heterozygous PRLR edited females can be produced by breeding homozygous PRLR edited males with wild type females. The cross will produce equal numbers of heterozygous edited males and females.

### Example 4: Prolactin Receptor Sequences

>NP_776580.1 prolactin receptor short isoform precursor [Bos taurus]
(SEQ ID NO: 1)
MKENAASRVVFILLLFLSVSLLNGQSPPEKPKLVKCRSPGKETFTCWWEPGADGGLPTNYTLTYHKEGETLIH
ECPDYKTGGPNSCYFSKKHTSIWKMYVITVNAINQMGISSSDPLYVHVTYIVEPEPPANLTLELKHPEDRKPY
LWIKWSPPTMTDVKSGWFIIQYEIRLKPEKATDWETHFTLKQTQLKIFNLYPGQKYLVQIRCKPDHGYWSEWS
PESSIQIPNDFPVKDTSMWIFVAILSAVICLIMVWAVALKGYSMVTCILPPVPGPKIKGFDVHLLEISQPSRL
VSMF
>NP_001034815.1 prolactin receptor long isoform precursor [Bos taurus]
(SEQ ID NO: 2)
MKENAASRVVFILLLFLSVSLLNGQSPPEKPKLVKCRSPGKETFTCWWEPGADGGLPTNYTLTYHKEGETLIH
ECPDYKTGGPNSCYFSKKHTSIWKMYVITVNAINQMGISSSDPLYVHVTYIVEPEPPANLTLELKHPEDRKPY
LWIKWSPPTMTDVKSGWFIIQYEIRLKPEKATDWETHFTLKQTQLKIFNLYPGQKYLVQIRCKPDHGYWSEWS
PESSIQIPNDFPVKDTSMWIFVAILSAVICLIMVWAVALKGYSMVTCILPPVPGPKIKGEDVHLLEKGKSEEL
LRALESQDFPPTSDCEDLLMEFIEVDDCEDQQLMPRPSKEHTEQGVKPMHLDLDSDSGRGSCDSPSLLSEKCD
EPQAHPSKFHTPEGPEKLENPETNLTCLQAPQSTSVEGKIPYFLANGPKSSTWPFPQPPSLYSPRYSYHNIAD
VCELALGMAGTTATSLDQTDQHALKASKTIETGREGKATKQRESEGCSSKPDQDTVWPRPQDKTPLISAKPLE
YVEIHKVSQDGVLALFPKQNEKFGAPEASKEYSKVSRVTDSNILVLVPDPQAQNLTLLEEPAKKAPPALP
>Mature prolactin receptor short isoform [Bos taurus]
(SEQ ID NO: 3)
QSPPEKPKLVKCRSPGKETFTCWWEPGADGGLPTNYTLTYHKEGETLIHECPDYKTGGPNSCYFSKKHTSIWK
MYVITVNAINQMGISSSDPLYVHVTYIVEPEPPANLTLELKHPEDRKPYLWIKWSPPTMTDVKSGWFIIQYEI
RLKPEKATDWETHFTLKQTQLKIFNLYPGQKYLVQIRCKPDHGYWSEWSPESSIQIPNDFPVKDTSMWIFVAI
LSAVICLIMVWAVALKGYSMVTCILPPVPGPKIKGEDVHLLEISQPSRLVSMF
>Mature prolactin receptor long isoform [Bos taurus]
(SEQ ID NO: 4)
QSPPEKPKLVKCRSPGKETFTCWWEPGADGGLPTNYTLTYHKEGETLIHECPDYKTGGPNSCYFSKKHTSIWK
MYVITVNAINQMGISSSDPLYVHVTYIVEPEPPANLTLELKHPEDRKPYLWIKWSPPTMTDVKSGWFIIQYEI
RLKPEKATDWETHFTLKQTQLKIFNLYPGQKYLVQIRCKPDHGYWSEWSPESSIQIPNDFPVKDTSMWIFVAI
LSAVICLIMVWAVALKGYSMVTCILPPVPGPKIKGFDVHLLEKGKSEELLRALESQDFPPTSDCEDLLMEFIE
VDDCEDQQLMPRPSKEHTEQGVKPMHLDLDSDSGRGSCDSPSLLSEKCDEPQAHPSKFHTPEGPEKLENPETN
LTCLQAPQSTSVEGKIPYFLANGPKSSTWPFPQPPSLYSPRYSYHNIADVCELALGMAGTTATSLDQTDQHAL
KASKTIETGREGKATKQRESEGCSSKPDQDTVWPRPQDKTPLISAKPLEYVEIHKVSQDGVLALFPKQNEKFG
APEASKEYSKVSRVTDSNILVLVPDPQAQNLTLLEEPAKKAPPALP
>Mature prolactin receptor short isoform with H188R substitution [Bos taurus]
(SEQ ID NO: 5)
QSPPEKPKLVKCRSPGKETFTCWWEPGADGGLPTNYTLTYHKEGETLIHECPDYKTGGPNSCYFSKKHTSIWK
MYVITVNAINQMGISSSDPLYVHVTYIVEPEPPANLTLELKHPEDRKPYLWIKWSPPTMTDVKSGWFIIQYEI
RLKPEKATDWETHFTLKQTQLKIFNLYPGQKYLVQIRCKPDRGYWSEWSPESSIQIPNDFPVKDTSMWIFVAI
LSAVICLIMVWAVALKGYSMVTCILPPVPGPKIKGFDVHLLEISQPSRLVSMF
>Mature prolactin receptor long isoform with H188R substitution [Bos taurus]
(SEQ ID NO: 6)
QSPPEKPKLVKCRSPGKETFTCWWEPGADGGLPTNYTLTYHKEGETLIHECPDYKTGGPNSCYFSKKHTSIWK
MYVITVNAINQMGISSSDPLYVHVTYIVEPEPPANLTLELKHPEDRKPYLWIKWSPPTMTDVKSGWFIIQYEI
RLKPEKATDWETHFTLKQTQLKIFNLYPGQKYLVQIRCKPDRGYWSEWSPESSIQIPNDFPVKDTSMWIFVAI
LSAVICLIMVWAVALKGYSMVTCILPPVPGPKIKGFDVHLLEKGKSEELLRALESQDFPPTSDCEDLLMEFIE
VDDCEDQQLMPRPSKEHTEQGVKPMHLDLDSDSGRGSCDSPSLLSEKCDEPQAHPSKFHTPEGPEKLENPETN
LTCLQAPQSTSVEGKIPYFLANGPKSSTWPFPQPPSLYSPRYSYHNIADVCELALGMAGTTATSLDQTDQHAL
KASKTIETGREGKATKQRESEGCSSKPDQDTVWPRPQDKTPLISAKPLEYVEIHKVSQDGVLALFPKQNEKFG
APEASKEYSKVSRVTDSNILVLVPDPQAQNLTLLEEPAKKAPPALP
>NM 174155.3 Bos taurus prolactin receptor (PRLR), transcript variant 1, mRNA
(SEQ ID NO: 7)
GGCAAATGCTGAGGATACTTTCCAAGTGAACCCTGAGTGAACCTCTAATATATTTATTTCCTGTGGAAAGAGG
AAGGAGCCAACATGAAGGAAAATGCAGCATCTAGAGTGGTTTTCATTTTGCTACTTTTTCTCAGTGTCAGCCT
TCTGAATGGACAGTCACCTCCTGAAAAACCCAAGCTCGTTAAATGTCGGTCTCCTGGAAAGGAAACATTCACC
TGCTGGTGGGAGCCTGGGGCAGATGGAGGACTTCCTACCAATTACACGCTGACTTACCACAAGGAAGGAGAAA
CACTCATCCATGAATGTCCAGACTACAAAACCGGGGGCCCCAACTCCTGCTACTTTAGCAAGAAGCACACCTC
CATATGGAAGATGTACGTCATCACAGTAAACGCCATCAACCAGATGGGAATCAGTTCCTCGGATCCACTTTAT
GTGCACGTGACTTACATAGTTGAACCAGAGCCTCCTGCAAACCTGACTTTGGAATTAAAACATCCAGAAGATA
GAAAACCATATCTATGGATAAAATGGTCTCCACCCACCATGACTGATGTAAAATCTGGTTGGTTCATTATCCA
GTACGAAATTCGATTAAAACCTGAGAAAGCAACTGATTGGGAGACTCATTTTACTCTGAAGCAAACTCAGCTT
AAGATTTTCAACTTATATCCAGGACAAAAATACCTTGTGCAGATTCGCTGCAAGCCAGACCATGGATACTGGA
GTGAGTGGAGCCCAGAGAGCTCCATCCAGATACCTAATGACTTCCCAGTGAAGGACACAAGCATGTGGATCTT
TGTGGCCATCCTTTCTGCTGTCATCTGTTTGATTATGGTCTGGGCAGTGGCTTTGAAGGGCTATAGCATGGTG
ACCTGCATCCTCCCACCAGTTCCAGGGCCAAAAATAAAAGGATTTGATGTTCATCTGCTGGAGATATCACAGC
CTTCTCGCCTTGTGTCTATGTTTTAATAGGAGGGCAAGTCCGAAGAACTTCTGCGAGCTCTGGAAAGCCAAGA
CTTCCCCCCCACTTCTGACTGCGAGGACTTGCTGATGGAGTTCATAGAGGTAGATGACTGTGAGGACCAGCAG
CTGATGCCACGCCCCTCCAAAGAACACACGGAGCAAGGCGTGAAGCCCATGCACCTGGATCTTGACAGTGACT
CTGGCCGGGGCAGCTGCGACAGCCCTTCGCTCTTGTCTGAAAAGTGTGATGAACCTCAGGCCCATCCCTCCAA
GTTCCATACTCCCGAGGGCCCTGAGAAGCTGGAGAATCCGGAAACAAACCTTACATGTCTCCAGGCCCCTCAG
AGCACAAGCGGGGAAGGCAAAATCCCCTATTTTCTGGCCAATGGACCCAAATCTTCCACATGGCCTTTCCCGC
AGCCCCCCAGCCTATACAGCCCCAGATATTCTTACCACAACATTGCTGACGTGTGTGAGCTGGCCCTGGGCAT
GGCCGGCACCACAGCCACTTCGCTGGACCAAACAGACCAACATGCTTTAAAAGCCTCAAAAACCATTGAAACT
GGCAGGGAAGGAAAGGCAACCAAGCAGAGGGAGTCAGAAGGCTGCAGTTCCAAGCCTGACCAAGACACGGTGT
GGCCACGACCCCAAGACAAAACCCCCTTGATCTCTGCTAAACCCTTGGAATACGTGGAGATCCACAAGGTCAG
CCAAGATGGAGTGCTGGCTCTGTTCCCAAAACAAAACGAGAAGTTTGGCGCCCCTGAAGCCAGCAAGGAGTAC
TCAAAGGTGTCCCGGGTGACAGATAGCAACATCCTGGTATTGGTGCCGGATCCGCAAGCGCAAAACCTGACTC
TGTTAGAAGAACCAGCCAAGAAGGCCCCGCCAGCCCTGCCATAGAATCCAGCCAAGGCCGACCTGGCTATCTC
CCCCACAACCCCAGGCAACTGCAGACTCCAGTTGGGCTGGGGACTGGGTCCCGCAGGTTTTATGCACTCTTGC
AGTGAGAGTTATGGAAGGATGGGTTCAATTGTGATTTTCCTTCAGGGAACACTACAGAGTACGTGAAATGCAC
TCTACCAGAGAGGGCTCAAGAACAGGGTTAGAATGACACTACCCAACTCCCAGTTCACTCTTAATTCTCTATT
TTCAACCAGTTGCCTCTTTGTCCAACAGCTGATTCCAGAACAAATCGTTCCATCTTGTGTGATTTGTAGATTT
ACTTTTTTGCTATTAGTTGTCAGATTATATGTTCAAAGATATAAAAGCACATTGCCTAGTATTCTTAAGAGAC
AGTGCCAATAGGTATATAATCTGGAAAAGGCCTTCATGGTTTCGTATGTGACAGAGGGGTATAAGTCAGTCAA
AATTGTTTACCATGGGAAGATGGTAGATAGGAGAGAAATGCCATGAAAACCACTTTGAAGACCAGTTGCTTAA
CCTTTGCACTCCTCTTT
>NM 001039726.2 Bos taurus prolactin receptor (PRLR), transcript variant 2, mRNA
(SEQ ID NO: 8)
GGCAAATGCTGAGGATACTTTCCAAGTGAACCCTGAGTGAACCTCTAATATATTTATTTCCTGTGGAAAGAGG
AAGGAGCCAACATGAAGGAAAATGCAGCATCTAGAGTGGTTTTCATTTTGCTACTTTTTCTCAGTGTCAGCCT
TCTGAATGGACAGTCACCTCCTGAAAAACCCAAGCTCGTTAAATGTCGGTCTCCTGGAAAGGAAACATTCACC
TGCTGGTGGGAGCCTGGGGCAGATGGAGGACTTCCTACCAATTACACGCTGACTTACCACAAGGAAGGAGAAA
CACTCATCCATGAATGTCCAGACTACAAAACCGGGGGCCCCAACTCCTGCTACTTTAGCAAGAAGCACACCTC
CATATGGAAGATGTACGTCATCACAGTAAACGCCATCAACCAGATGGGAATCAGTTCCTCGGATCCACTTTAT
GTGCACGTGACTTACATAGTTGAACCAGAGCCTCCTGCAAACCTGACTTTGGAATTAAAACATCCAGAAGATA
GAAAACCATATCTATGGATAAAATGGTCTCCACCCACCATGACTGATGTAAAATCTGGTTGGTTCATTATCCA
GTACGAAATTCGATTAAAACCTGAGAAAGCAACTGATTGGGAGACTCATTTTACTCTGAAGCAAACTCAGCTT
AAGATTTTCAACTTATATCCAGGACAAAAATACCTTGTGCAGATTCGCTGCAAGCCAGACCATGGATACTGGA
GTGAGTGGAGCCCAGAGAGCTCCATCCAGATACCTAATGACTTCCCAGTGAAGGACACAAGCATGTGGATCTT
TGTGGCCATCCTTTCTGCTGTCATCTGTTTGATTATGGTCTGGGCAGTGGCTTTGAAGGGCTATAGCATGGTG
ACCTGCATCCTCCCACCAGTTCCAGGGCCAAAAATAAAAGGATTTGATGTTCATCTGCTGGAGAAGGGCAAGT
CCGAAGAACTTCTGCGAGCTCTGGAAAGCCAAGACTTCCCCCCCACTTCTGACTGCGAGGACTTGCTGATGGA
GTTCATAGAGGTAGATGACTGTGAGGACCAGCAGCTGATGCCACGCCCCTCCAAAGAACACACGGAGCAAGGC
GTGAAGCCCATGCACCTGGATCTTGACAGTGACTCTGGCCGGGGCAGCTGCGACAGCCCTTCGCTCTTGTCTG
AAAAGTGTGATGAACCTCAGGCCCATCCCTCCAAGTTCCATACTCCCGAGGGCCCTGAGAAGCTGGAGAATCC
GGAAACAAACCTTACATGTCTCCAGGCCCCTCAGAGCACAAGCGTGGAAGGCAAAATCCCCTATTTTCTGGCC
AATGGACCCAAATCTTCCACATGGCCTTTCCCGCAGCCCCCCAGCCTATACAGCCCCAGATATTCTTACCACA
ACATTGCTGACGTGTGTGAGCTGGCCCTGGGCATGGCCGGCACCACAGCCACTTCGCTGGACCAAACAGACCA
ACATGCTTTAAAAGCCTCAAAAACCATTGAAACTGGCAGGGAAGGAAAGGCAACCAAGCAGAGGGAGTCAGAA
GGCTGCAGTTCCAAGCCTGACCAAGACACGGTGTGGCCACGACCCCAAGACAAAACCCCCTTGATCTCTGCTA
AACCCTTGGAATACGTGGAGATCCACAAGGTCAGCCAAGATGGAGTGCTGGCTCTGTTCCCAAAACAAAACGA
GAAGTTTGGCGCCCCTGAAGCCAGCAAGGAGTACTCAAAGGTGTCCCGGGTGACAGATAGCAACATCCTGGTA
TTGGTGCCGGATCCGCAAGCGCAAAACCTGACTCTGTTAGAAGAACCAGCCAAGAAGGCCCCGCCAGCCCTGC
CATAGAATCCAGCCAAGGCCGACCTGGCTATCTCCCCCACAACCCCAGGCAACTGCAGACTCCAGTTGGGCTG
GGGACTGGGTCCCGCAGGTTTTATGCACTCTTGCAGTGAGAGTTATGGAAGGATGGGTTCAATTGTGATTTTC
CTTCAGGGAACACTACAGAGTACGTGAAATGCACTCTACCAGAGAGGGCTCAAGAACAGGGTTAGAATGACAC
TACCCAACTCCCAGTTCACTCTTAATTCTCTATTTTCAACCAGTTGCCTCTTTGTCCAACAGCTGATTCCAGA
ACAAATCGTTCCATCTTGTGTGATTTGTAGATTTACTTTTTTGCTATTAGTTGTCAGATTATATGTTCAAAGA
TATAAAAGCACATTGCCTAGTATTCTTAAGAGACAGTGCCAATAGGTATATAATCTGGAAAAGGCCTTCATGG
TTTCGTATGTGACAGAGGGGTATAAGTCAGTCAAAATTGTTTACCATGGGAAGATGGTAGATAGGAGAGAAAT
GCCATGAAAACCACTTTGAAGACCAGTTGCTTAACCTTTGCACTCCTCTTT

### Example 5: Comparison of Editing Frequencies of PRLR sgRNAs with S. Pyogenes (Spy) Cas9 and S. Thermophilus (Stherm) Cas9

The editing frequencies of PRLR_H188R_4 (SEQ ID NO: 12) sgRNPs assembled with Spy Cas9 or Stherm Cas9 were compared. Bovine zygotes were injected with either sgRNP between 16-18 hours post insemination and allowed to grow to blastocyst stage at Day 7 post insemination. Blastocysts were sequenced to determine editing frequency.

### Editing Reagents Preparation

### Standard Injection Mix

sgRNA PRLR_H188R_4 (SEQ ID NO: 12) was incubated at 95° C. for 2 minutes and brought to Room Temperature (RT). The following reagents were combined: 0.9 μL water, 0.64 μL Cas9 stock (10 mg/mL stock), and 2.92 μL sgRNA (PRLR_H188R_4 (SEQ ID NO: 12) at 1500 ng/mL) to obtain a solution volume of 4.46 μL. The mixture was incubated at RT for 10 minutes. 228 μL TE buffer (low EDTA, TE_{low}) was added to 2.23 μL of the mixture to obtain a Reagent/TE_{low} mix.

27 μL of Reagent/TE_{low} mix was combined with 3 μL of HDR template (PRLR HDR 50 nucleotide arms-top strand (SEQ ID NO: 14). HDR stock was 250 ng/mL in TE_{low} buffer). This mixture was centrifuged at 8000×g for 1 minute prior to injecting into zygotes.

### Zygote Injection

Bovine zygotes were stripped of the cumulus cells at about 15 hours post insemination. Zygotes were injected with the editing reagents between 16-18 hours post insemination using an Eppendorf Femtoj et injector with settings of Pi=200, time=0.25 sec and Pc=15. Zygotes were cultured in BO-IVC medium (InVitro Bioscience; Cornwall, UK) for 7 days and blastocyst stage embryos were collected as samples for Illumina sequencing to determine how many embryos had sequence reads for both PRLR edited and WT reads.

### Results

| | Spy Cas9 | Stherm Cas9 |
|---|---|---|
| # Blastocysts ≥ 10% | 6 | 3 |
| Desired Edit | | |
| # Edited Blastocysts with | 0 | 0 |
| WT reads | | |
| Total Edited Blastocysts | 25 | 10 |

These results demonstrate mammalian blastocyst stage embryos harboring a modification of the histidine residue at position 188 of the PRLR protein could be successfully obtained via CRISPR/Cas gene editing.

### Example 6: PRLR Titration Injection

Previous bovine zygote injections of editing reagents for making an edit in the Prolactin Receptor (PRLR) gene have resulted in editing of all embryos analyzed. The precise edit of interest results in a heterozygous edit status where one allele for PRLR is edited and the other is wild type (WT; not edited). To achieve a heterozygous state for PRLR editing, the previous preparations of editing reagents was diluted to see if an increase in WT reads can be obtained while also achieving the desired edit.

### Editing Reagents Preparation

### Standard Injection Mix (1× Mix)

sgRNA PRLR_H188R_4 (SEQ ID NO: 12) was incubated at 95° C. for 2 minutes and brought to Room Temperature (RT). The following reagents were combined: 0.9 μL water, 0.64 μL Cas9 protein stock (10 mg/mL stock) and 2.92 μL sgRNA (PRLR_H188R_4 (SEQ ID NO: 12) at 1500 ng/mL) to obtain a solution volume of 4.46 μL. The mixture was incubated at RT for 10 minutes. 228 μL TE buffer (low EDTA, TE_{low}) was added to 2.23 μL of the mixture to obtain a Reagent/TE_{low} mix.

27 μL of Reagent/TE_{low} mix was combined with 3 μL of HDR template (PRLR HDR 50 nucleotide arms-top strand (SEQ ID NO: 14). HDR stock is 250 ng/mL in TE_{low} buffer). This mixture was centrifuged at 8000×g for 1 minute prior to injecting into zygotes. This mixture represents 1× Mix of the editing reagents.

The 1× Mix was diluted in half by adding equal volumes of TE_{low} buffer and the 1× Mix. This mixture represents the ½× Mix. The ½× Mix was diluted in half by adding equal volumes of TE_{low} buffer and the ½× Mix. This represents the ¼× Mix.

### Zygote Injection

Bovine zygotes were stripped of the cumulus cells at about 15 hours post insemination. Zygotes were injected with the editing reagents between 16-18 hours post insemination. Zygotes were cultured in BO-IVC medium (InVitro Bioscience; Cornwall, UK) for 7 days and blastocyst stage embryos were collected as samples for Illumina sequencing to determine how many embryos had sequence reads for both PRLR edited and WT reads.

### Results

| | 1X Mix | ½X Mix | ¼X Mix |
|---|---|---|---|
| # Blastocysts ≥ 10% | 0 | 5 | 7 |
| WT | | | |
| Total Edited | 5 | 17 | 12 |
| Blastocysts | | | |
| % Edited | 0% | 29% | 58% |
| Blastocysts with WT | | | |
| reads | | | |

These results demonstrate mammalian blastocyst stage embryos harboring a WT PRLR allele and an allele with a H188 modification in the PRLR protein could be successfully obtained via CRISPR/Cas gene editing.

### EXEMPLARY EMBODIMENTS

The present disclosure may be described in terms of the following non-limiting embodiments:

Embodiment 1: A nonhuman mammal or progeny thereof comprising an edited chromosomal sequence that alters expression or activity of a prolactin receptor (PRLR) protein, wherein the mammal produces milk without having been pregnant.

Embodiment 2: The mammal or progeny of Embodiment 1, wherein the edited chromosomal sequence encodes a modification of one or more amino acids corresponding to positions 105 to 205 of the PRLR protein of SEQ ID NO: 3 or 4.

Embodiment 3: The mammal or progeny of Embodiment 1 or 2, wherein the edited chromosomal sequence encodes a modification corresponding to the histidine residue at position 188 of the PRLR protein of SEQ ID NO: 3 or 4.

Embodiment 4: The mammal or progeny of Embodiment 2 or 3, wherein the edited chromosomal sequence encodes a histidine to arginine substitution corresponding to the histidine residue at position 188 of the PRLR protein of SEQ ID NO: 3 or 4.

Embodiment 5: The mammal or progeny of any one of Embodiments 1-4, wherein the edited chromosomal sequence results in reduced or no functional PRLR protein activity.

Embodiment 6: The mammal or progeny of any one of Embodiments 1-5, wherein the edited chromosomal sequence comprises any one of SEQ ID NOs: 14-17.

Embodiment 7: The mammal or progeny of any one of Embodiments 1-6, wherein the edited chromosomal sequence comprises SEQ ID NO: 14.

Embodiment 8: The mammal or progeny of any one of Embodiments 1-7, wherein the mammal is a dairy animal selected from a cow, a heifer, a goat, a sheep, a camel, a donkey, a horse, a reindeer, a bison, a water buffalo, a moose, and a yak.

Embodiment 9: The mammal or progeny of any one of Embodiments 1-7, wherein the mammal is a bovine.

Embodiment 10: The mammal or progeny of any one of Embodiments 1-7, wherein the mammal is dairy animal selected from a cow, a heifer, a goat, and a sheep.

Embodiment 11: The mammal or progeny of any one of Embodiments 1-7, wherein the mammal is a cow or heifer.

Embodiment 12: A nonhuman mammalian cell, the cell comprising an edited chromosomal sequence that alters expression or activity of a prolactin receptor (PRLR) protein, wherein the edited chromosomal sequence encodes a modification of one or more amino acids corresponding to positions 105 to 205 of the PRLR protein of SEQ ID NO: 3 or 4.

Embodiment 13: The mammalian cell of Embodiment 12, wherein the edited chromosomal sequence encodes a modification corresponding to the histidine residue at position 188 of the PRLR protein of SEQ ID NO: 3 or 4.

Embodiment 14: A method of generating a nonhuman mammal that produces milk without being pregnant, the method comprising: editing a chromosomal sequence that alters expression or activity of a PRLR protein, wherein the edited chromosomal sequence encodes a modification of one or more amino acids corresponding to positions 105 to 205 of the PRLR protein of SEQ ID NO: 3 or 4.

Embodiment 15: The method of Embodiment 14, wherein the edited chromosomal sequence encodes a modification corresponding to the histidine residue at position 188 of the PRLR protein of SEQ ID NO: 3 or 4.

Embodiment 16: The method of Embodiment 14 or 15, wherein the edited chromosomal sequence encodes a histidine to arginine substitution corresponding to the histidine residue at position 188 of the PRLR protein of SEQ ID NO: 3 or 4.

Embodiment 17: The method of any one of Embodiments 14-16, wherein the edited chromosomal sequence results in reduced or no functional PRLR protein activity.

Embodiment 18: The method of any one of Embodiments 14-17, wherein the editing comprises use of a CRISPR system comprising a gRNA comprising a sequence of any one of SEQ ID NO: 9-13 and an HDR repair template comprising a sequence of any one of SEQ ID NO: 14-17.

Embodiment 19: The method of any one of Embodiments 14-17, wherein the editing comprises use of a CRISPR system comprising a gRNA comprising SEQ ID NO: 12 and an HDR template comprising SEQ ID NO: 14.

Embodiment 20: The method of any one of Embodiments 14-17, wherein the editing comprises use of a PRLR guide RNA comprising any one of SEQ ID NO: 9-13.

Embodiment 21: The method of any one of Embodiments 14-17, wherein the editing comprises the use of a PRLR guide RNA comprising SEQ ID NO: 12.

Embodiment 22: A genetically modified Bos taurus comprising a prolactin receptor gene comprising an edited chromosomal sequence encoding a modification of one or more amino acids from position 105 to 205 of the PRLR protein of SEQ ID NO: 3 or 4.

Embodiment 23: The genetically modified Bos taurus of Embodiment 22, wherein the edited chromosomal sequence encodes a modification of the histidine residue at position 188 of the PRLR protein.

Embodiment 24: The genetically modified Bos taurus of Embodiment 22 or 23, wherein the edited chromosomal sequence encodes a substitution of the histidine residue at position 188 of the PRLR protein with an arginine residue.

Embodiment 25: The genetically modified Bos taurus of any one of Embodiments 22-24, wherein the edited chromosomal sequence comprises any one of SEQ ID NOs: 14-17.

Embodiment 26: The genetically modified Bos taurus any one of Embodiments 22-25, wherein the edited chromosomal sequence comprises SEQ ID NO: 14.

Embodiment 27: The genetically modified Bos taurus of any one of Embodiments 22-26, wherein the edited chromosomal sequence results in reduced or no functional PRLR protein activity.

Embodiment 28: The genetically modified Bos taurus of any one of Embodiments 22-27, wherein the genetically modified Bos taurus is a cow or heifer that produces milk without having been pregnant.

Embodiment 29: A gRNA comprising a sequence of any one of SEQ ID NO: 9-13.

Embodiment 30: A kit comprising a gRNA that specifically hybridizes to a prolactin receptor (PRLR) gene, a homology directed repair (HDR) template comprising any one of SEQ ID NOs: 14-17, and instructions for use.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention. Examples disclosed herein are provided by way of exemplification and are not intended to limit the scope of the invention.

What is claimed is:

1. A non-human mammal or progeny thereof comprising an edited chromosomal sequence that alters expression or activity of a prolactin receptor (PRLR) protein, wherein the non-human mammal produces milk without having been pregnant and wherein the edited chromosomal sequence encodes a modification of one or more amino acids corresponding to positions 105 to 205 of the PRLR protein of SEQ ID NO: 3 or 4.

2. (canceled)

3. The non-human mammal or progeny of claim 1, wherein the edited chromosomal sequence encodes a modification corresponding to the histidine residue at position 188 of the PRLR protein of SEQ ID NO: 3 or 4.

4. The non-human mammal or progeny of claim 1, wherein the edited chromosomal sequence encodes a histidine to arginine substitution corresponding to the histidine residue at position 188 of the PRLR protein of SEQ ID NO: 3 or 4.

5. The nonhuman mammal or progeny of claim 1, wherein the edited chromosomal sequence results in reduced or no functional PRLR protein activity.

6. The non-human mammal or progeny of claim 1, wherein the edited chromosomal sequence comprises any one of SEQ ID NOs: 14-17.

7. The non-human mammal or progeny of claim 1, wherein the edited chromosomal sequence comprises SEQ ID NO: 14.

8. (canceled)

9. The non-human mammal or progeny of claim 1, wherein the non-human mammal is a bovine.

10. The non-human mammal or progeny of claim 1, wherein the non-human mammal is a dairy animal selected from a cow, a heifer, a goat, and a sheep.

11. The non-human mammal or progeny of claim 1, wherein the non-human mammal is a cow or a heifer.

12. A non-human mammalian cell, the cell comprising an edited chromosomal sequence that alters expression or activity of a prolactin receptor (PRLR) protein, wherein the edited chromosomal sequence encodes a modification of one or more amino acids corresponding to positions 105 to 205 of the PRLR protein of SEQ ID NO: 3 or 4.

13. The non-human mammalian cell of claim 12, wherein the edited chromosomal sequence encodes a modification corresponding to the histidine residue at position 188 of the PRLR protein of SEQ ID NO: 3 or 4.

14. A method of generating a non-human mammal that produces milk without being pregnant, the method comprising:
editing a chromosomal sequence that reduces or eliminates expression or activity of a PRLR protein, wherein the edited chromosomal sequence encodes a modification of one or more amino acids corresponding to positions 105 to 205 of the PRLR protein of SEQ ID NO: 3 or 4.

15. The method of claim 14, wherein the edited chromosomal sequence encodes a modification corresponding to the histidine residue at position 188 of the PRLR protein of SEQ ID NO: 3 or 4.

16. The method of claim 14, wherein the edited chromosomal sequence encodes a histidine to arginine substitution corresponding to the histidine residue at position 188 of the PRLR protein of SEQ ID NO: 3 or 4.

17. (canceled)

18. The method of claim 14, wherein the editing comprises use of a CRISPR system comprising a gRNA comprising a sequence of any one of SEQ ID NO: 9-13 and an HDR repair template comprising a sequence of any one of SEQ ID NO: 14-17.

19. The method of claim 14, wherein the editing comprises use of a CRISPR system comprising a gRNA comprising SEQ ID NO: 12 and an HDR template comprising SEQ ID NO: 14.

20-30. (canceled)

31. The non-human mammal or progeny of claim 1, wherein the non-human mammal or progeny is a Bos taurus animal.

32. The non-human mammal or progeny of claim 6, wherein the non-human mammal or progeny is a Bos taurus animal.

33. The Bos taurus of claim 32, wherein the Bos taurus animal is a cow or heifer that produces milk without getting pregnant.

34. A non-human mammal produced through nuclear transfer of the non-human mammalian cell of claim 12.


SEQUENCE LISTING (17 sequences)

SEQ ID NO: 1 - protein, 296 aa, Bos taurus
MKENAASRVVFILLLFLSVSLLNGQSPPEKPKLVKCRSPGKETFTCWWEPGADGGLPTNYTLTYHKEGETLIHECPDYKTGGPNSCYFSKKHTSIWKMYVITVNAINQMGISSSDPLYVHVTYIVEPEPPANLTLELKHPEDRKPYLWIKWSPPTMTDVKSGWFIIQYEIRLKPEKATDWETHFTLKQTQLKIFNLYPGQKYLVQIRCKPDHGYWSEWSPESSIQIPNDFPVKDTSMWIFVAILSAVICLIMVWAVALKGYSMVTCILPPVPGPKIKGFDVHLLEISQPSRLVSMF

SEQ ID NO: 2 - protein, 581 aa, Bos taurus
MKENAASRVVFILLLFLSVSLLNGQSPPEKPKLVKCRSPGKETFTCWWEPGADGGLPTNYTLTYHKEGETLIHECPDYKTGGPNSCYFSKKHTSIWKMYVITVNAINQMGISSSDPLYVHVTYIVEPEPPANLTLELKHPEDRKPYLWIKWSPPTMTDVKSGWFIIQYEIRLKPEKATDWETHFTLKQTQLKIFNLYPGQKYLVQIRCKPDHGYWSEWSPESSIQIPNDFPVKDTSMWIFVAILSAVICLIMVWAVALKGYSMVTCILPPVPGPKIKGFDVHLLEKGKSEELLRALESQDFPPTSDCEDLLMEFIEVDDCEDQQLMPRPSKEHTEQGVKPMHLDLDSDSGRGSCDSPSLLSEKCDEPQAHPSKFHTPEGPEKLENPETNLTCLQAPQSTSVEGKIPYFLANGPKSSTWPFPQPPSLYSPRYSYHNIADVCELALGMAGTTATSLDQTDQHALKASKTIETGREGKATKQRESEGCSSKPDQDTVWPRPQDKTPLISAKPLEYVEIHKVSQDGVLALFPKQNEKFGAPEASKEYSKVSRVTDSNILVLVPDPQAQNLTLLEEPAKKAPPALP

SEQ ID NO: 3 - protein, 272 aa, Bos taurus
QSPPEKPKLVKCRSPGKETFTCWWEPGADGGLPTNYTLTYHKEGETLIHECPDYKTGGPNSCYFSKKHTSIWKMYVITVNAINQMGISSSDPLYVHVTYIVEPEPPANLTLELKHPEDRKPYLWIKWSPPTMTDVKSGWFIIQYEIRLKPEKATDWETHFTLKQTQLKIFNLYPGQKYLVQIRCKPDHGYWSEWSPESSIQIPNDFPVKDTSMWIFVAILSAVICLIMVWAVALKGYSMVTCILPPVPGPKIKGFDVHLLEISQPSRLVSMF

SEQ ID NO: 4 - protein, 557 aa, Bos taurus
QSPPEKPKLVKCRSPGKETFTCWWEPGADGGLPTNYTLTYHKEGETLIHECPDYKTGGPNSCYFSKKHTSIWKMYVITVNAINQMGISSSDPLYVHVTYIVEPEPPANLTLELKHPEDRKPYLWIKWSPPTMTDVKSGWFIIQYEIRLKPEKATDWETHFTLKQTQLKIFNLYPGQKYLVQIRCKPDHGYWSEWSPESSIQIPNDFPVKDTSMWIFVAILSAVICLIMVWAVALKGYSMVTCILPPVPGPKIKGFDVHLLEKGKSEELLRALESQDFPPTSDCEDLLMEFIEVDDCEDQQLMPRPSKEHTEQGVKPMHLDLDSDSGRGSCDSPSLLSEKCDEPQAHPSKFHTPEGPEKLENPETNLTCLQAPQSTSVEGKIPYFLANGPKSSTWPFPQPPSLYSPRYSYHNIADVCELALGMAGTTATSLDQTDQHALKASKTIETGREGKATKQRESEGCSSKPDQDTVWPRPQDKTPLISAKPLEYVEIHKVSQDGVLALFPKQNEKFGAPEASKEYSKVSRVTDSNILVLVPDPQAQNLTLLEEPAKKAPPALP

SEQ ID NO: 5 - protein, 272 aa, Bos taurus
QSPPEKPKLVKCRSPGKETFTCWWEPGADGGLPTNYTLTYHKEGETLIHECPDYKTGGPNSCYFSKKHTSIWKMYVITVNAINQMGISSSDPLYVHVTYIVEPEPPANLTLELKHPEDRKPYLWIKWSPPTMTDVKSGWFIIQYEIRLKPEKATDWETHFTLKQTQLKIFNLYPGQKYLVQIRCKPDRGYWSEWSPESSIQIPNDFPVKDTSMWIFVAILSAVICLIMVWAVALKGYSMVTCILPPVPGPKIKGFDVHLLEISQPSRLVSMF

SEQ ID NO: 6 - protein, 557 aa, Bos taurus
QSPPEKPKLVKCRSPGKETFTCWWEPGADGGLPTNYTLTYHKEGETLIHECPDYKTGGPNSCYFSKKHTSIWKMYVITVNAINQMGISSSDPLYVHVTYIVEPEPPANLTLELKHPEDRKPYLWIKWSPPTMTDVKSGWFIIQYEIRLKPEKATDWETHFTLKQTQLKIFNLYPGQKYLVQIRCKPDRGYWSEWSPESSIQIPNDFPVKDTSMWIFVAILSAVICLIMVWAVALKGYSMVTCILPPVPGPKIKGFDVHLLEKGKSEELLRALESQDFPPTSDCEDLLMEFIEVDDCEDQQLMPRPSKEHTEQGVKPMHLDLDSDSGRGSCDSPSLLSEKCDEPQAHPSKFHTPEGPEKLENPETNLTCLQAPQSTSVEGKIPYFLANGPKSSTWPFPQPPSLYSPRYSYHNIADVCELALGMAGTTATSLDQTDQHALKASKTIETGREGKATKQRESEGCSSKPDQDTVWPRPQDKTPLISAKPLEYVEIHKVSQDGVLALFPKQNEKFGAPEASKEYSKVSRVTDSNILVLVPDPQAQNLTLLEEPAKKAPPALP

SEQ ID NO: 7 - DNA, 2426 bp, Bos taurus
ggcaaatgctgaggatactttccaagtgaaccctgagtgaacctctaatatatttatttcctgtggaaagaggaaggagccaacatgaaggaaaatgcagcatctagagtggttttcattttgctactttttctcagtgtcagccttctgaatggacagtcacctcctgaaaaacccaagctcgttaaatgtcggtctcctggaaaggaaacattcacctgctggtgggagcctggggcagatggaggacttcctaccaattacacgctgacttaccacaaggaaggagaaacactcatccatgaatgtccagactacaaaaccgggggccccaactcctgctactttagcaagaagcacacctccatatggaagatgtacgtcatcacagtaaacgccatcaaccagatgggaatcagttcctcggatccactttatgtgcacgtgacttacatagttgaaccagagcctcctgcaaacctgactttggaattaaaacatccagaagatagaaaaccatatctatggataaaatggtctccacccaccatgactgatgtaaaatctggttggttcattatccagtacgaaattcgattaaaacctgagaaagcaactgattgggagactcattttactctgaagcaaactcagcttaagattttcaacttatatccaggacaaaaataccttgtgcagattcgctgcaagccagaccatggatactggagtgagtggagcccagagagctccatccagatacctaatgacttcccagtgaaggacacaagcatgtggatctttgtggccatcctttctgctgtcatctgtttgattatggtctgggcagtggctttgaagggctatagcatggtgacctgcatcctcccaccagttccagggccaaaaataaaaggatttgatgttcatctgctggagatatcacagccttctcgccttgtgtctatgttttaataggagggcaagtccgaagaacttctgcgagctctggaaagccaagacttcccccccacttctgactgcgaggacttgctgatggagttcatagaggtagatgactgtgaggaccagcagctgatgccacgcccctccaaagaacacacggagcaaggcgtgaagcccatgcacctggatcttgacagtgactctggccggggcagctgcgacagcccttcgctcttgtctgaaaagtgtgatgaacctcaggcccatccctccaagttccatactcccgagggccctgagaagctggagaatccggaaacaaaccttacatgtctccaggcccctcagagcacaagcggggaaggcaaaatcccctattttctggccaatggacccaaatcttccacatggcctttcccgcagccccccagcctatacagccccagatattcttaccacaacattgctgacgtgtgtgagctggccctgggcatggccggcaccacagccacttcgctggaccaaacagaccaacatgctttaaaagcctcaaaaaccattgaaactggcagggaaggaaaggcaaccaagcagagggagtcagaaggctgcagttccaagcctgaccaagacacggtgtggccacgaccccaagacaaaacccccttgatctctgctaaacccttggaatacgtggagatccacaaggtcagccaagatggagtgctggctctgttcccaaaacaaaacgagaagtttggcgcccctgaagccagcaaggagtactcaaaggtgtcccgggtgacagatagcaacatcctggtattggtgccggatccgcaagcgcaaaacctgactctgttagaagaaccagccaagaaggccccgccagccctgccatagaatccagccaaggccgacctggctatctcccccacaaccccaggcaactgcagactccagttgggctggggactgggtcccgcaggttttatgcactcttgcagtgagagttatggaaggatgggttcaattgtgattttccttcagggaacactacagagtacgtgaaatgcactctaccagagagggctcaagaacagggttagaatgacactacccaactcccagttcactcttaattctctattttcaaccagttgcctctttgtccaacagctgattccagaacaaatcgttccatcttgtgtgatttgtagatttacttttttgctattagttgtcagattatatgttcaaagatataaaagcacattgcctagtattcttaagagacagtgccaataggtatataatctggaaaaggccttcatggtttcgtatgtgacagaggggtataagtcagtcaaaattgtttaccatgggaagatggtagataggagagaaatgccatgaaaaccactttgaagaccagttgcttaacctttgcactcctcttt

SEQ ID NO: 8 - DNA, 2387 bp, Bos taurus
ggcaaatgctgaggatactttccaagtgaaccctgagtgaacctctaatatatttatttcctgtggaaagaggaaggagccaacatgaaggaaaatgcagcatctagagtggttttcattttgctactttttctcagtgtcagccttctgaatggacagtcacctcctgaaaaacccaagctcgttaaatgtcggtctcctggaaaggaaacattcacctgctggtgggagcctggggcagatggaggacttcctaccaattacacgctgacttaccacaaggaaggagaaacactcatccatgaatgtccagactacaaaaccgggggccccaactcctgctactttagcaagaagcacacctccatatggaagatgtacgtcatcacagtaaacgccatcaaccagatgggaatcagttcctcggatccactttatgtgcacgtgacttacatagttgaaccagagcctcctgcaaacctgactttggaattaaaacatccagaagatagaaaaccatatctatggataaaatggtctccacccaccatgactgatgtaaaatctggttggttcattatccagtacgaaattcgattaaaacctgagaaagcaactgattgggagactcattttactctgaagcaaactcagcttaagattttcaacttatatccaggacaaaaataccttgtgcagattcgctgcaagccagaccatggatactggagtgagtggagcccagagagctccatccagatacctaatgacttcccagtgaaggacacaagcatgtggatctttgtggccatcctttctgctgtcatctgtttgattatggtctgggcagtggctttgaagggctatagcatggtgacctgcatcctcccaccagttccagggccaaaaataaaaggatttgatgttcatctgctggagaagggcaagtccgaagaacttctgcgagctctggaaagccaagacttcccccccacttctgactgcgaggacttgctgatggagttcatagaggtagatgactgtgaggaccagcagctgatgccacgcccctccaaagaacacacggagcaaggcgtgaagcccatgcacctggatcttgacagtgactctggccggggcagctgcgacagcccttcgctcttgtctgaaaagtgtgatgaacctcaggcccatccctccaagttccatactcccgagggccctgagaagctggagaatccggaaacaaaccttacatgtctccaggcccctcagagcacaagcgtggaaggcaaaatcccctattttctggccaatggacccaaatcttccacatggcctttcccgcagccccccagcctatacagccccagatattcttaccacaacattgctgacgtgtgtgagctggccctgggcatggccggcaccacagccacttcgctggaccaaacagaccaacatgctttaaaagcctcaaaaaccattgaaactggcagggaaggaaaggcaaccaagcagagggagtcagaaggctgcagttccaagcctgaccaagacacggtgtggccacgaccccaagacaaaacccccttgatctctgctaaacccttggaatacgtggagatccacaaggtcagccaagatggagtgctggctctgttcccaaaacaaaacgagaagtttggcgcccctgaagccagcaaggagtactcaaaggtgtcccgggtgacagatagcaacatcctggtattggtgccggatccgcaagcgcaaaacctgactctgttagaagaaccagccaagaaggccccgccagccctgccatagaatccagccaaggccgacctggctatctcccccacaaccccaggcaactgcagactccagttgggctggggactgggtcccgcaggttttatgcactcttgcagtgagagttatggaaggatgggttcaattgtgattttccttcagggaacactacagagtacgtgaaatgcactctaccagagagggctcaagaacagggttagaatgacactacccaactcccagttcactcttaattctctattttcaaccagttgcctctttgtccaacagctgattccagaacaaatcgttccatcttgtgtgatttgtagatttacttttttgctattagttgtcagattatatgttcaaagatataaaagcacattgcctagtattcttaagagacagtgccaataggtatataatctggaaaaggccttcatggtttcgtatgtgacagaggggtataagtcagtcaaaattgtttaccatgggaagatggtagataggagagaaatgccatgaaaaccactttgaagaccagttgcttaacctttgcactcctcttt

SEQ ID NO: 9 - DNA, 20 bp, Bos taurus
attcgctgcaagccagacca

SEQ ID NO: 10 - DNA, 20 bp, Bos taurus
tcactccagtatccatggtc

SEQ ID NO: 11 - DNA, 20 bp, Bos taurus
tccactcactccagtatcca

SEQ ID NO: 12 - DNA, 20 bp, Bos taurus
gcaagccagaccatggatac

SEQ ID NO: 13 - DNA, 20 bp, Bos taurus
accatggatactggagtgag

SEQ ID NO: 14 - DNA, 101 bp, Bos taurus
cttatatccaggacaaaaataccttgtgcagattcgctgcaagccagaccgtggatactggagtgagtggagcccagagagctccatccagatacctaatg

SEQ ID NO: 15 - DNA, 101 bp, Bos taurus
cattaggtatctggatggagctctctgggctccactcactccagtatccacggtctggcttgcagcgaatctgcacaaggtatttttgtcctggatataag

SEQ ID NO: 16 - DNA, 151 bp, Bos taurus
agcaaactcagcttaagattttcaacttatatccaggacaaaaataccttgtgcagattcgctgcaagccagaccgtggatactggagtgagtggagcccagagagctccatccagatacctaatggtaagtgtcttgagattccccaagg

SEQ ID NO: 17 - DNA, 151 bp, Bos taurus
ccttggggaatctcaagacacttaccattaggtatctggatggagctctctgggctccactcactccagtatccacggtctggcttgcagcgaatctgcacaaggtatttttgtcctggatataagttgaaaatcttaagctgagtttgct